SEQUENCE LISTING (7 sequences)

SEQ ID NO: 1 - DNA, 21 bp, Artificial Sequence
  Description of Artificial Sequence Synthetic oligonucleotide
  Description of Combined DNA/RNA Molecule Synthetic oligonucleotide
cgggauaaguacucaaagatt

SEQ ID NO: 2 - RNA, 21 nt, Artificial Sequence
  Description of Artificial Sequence Synthetic oligonucleotide
ucuuugaguacuuaucccgga

SEQ ID NO: 3 - RNA, 21 nt, Artificial Sequence
  Description of Artificial Sequence Synthetic oligonucleotide
cgggauaaguacucaaagauu

SEQ ID NO: 4 - DNA, 1833 bp, Homo sapiens
ggtgagcgactaacgctagaaacagtggtgcgcggagaggagaggcctcgggatgtctctggcagatgagctcttagctgatctcgaagaggcagcagaagaggaggaaggaggaagctatggggaggaagaagaggagccagcgatcgaggatgtgcaggaggagacacagctggatctttccggggattcagtcaagaccatcgccaagctatgggatagtaagatgtttgctgagattatgatgaagattgaggagtatatcagcaagcaagccaaagcttcagaagtgatgggaccagtggaggccgcgcctgaataccgcgtcatcgtggatgccaacaacctgaccgtggagatcgaaaacgagctgaacatcatccataagttcatccgggataagtactcaaagagattccctgaactggagtccttggtccccaatgcactggattacatccgcacggtcaaggagctgggcaacagcctggacaagtgcaagaacaatgagaacctgcagcagatcctcaccaatgccaccatcatggtcgtcagcgtcaccgcctccaccacccaggggcagcagctgtcggaggaggagctggagcggctggaggaggcctgcgacatggcgctggagctgaacgcctccaagcaccgcatctacgagtatgtggagtcccggatgtccttcatcgcacccaacctgtccatcattatcggggcatccacggccgccaagatcatgggtgtggccggcggcctgaccaacctctccaagatgcccgcctgcaacatcatgctgctcggggcccagcgcaagacgctgtcgggcttctcgtctacctcagtgctgccccacaccggctacatctaccacagtgacatcgtgcagtccctgccaccggatctgcggcggaaagcggcccggctggtggccgccaagtgcacactggcagcccgtgtggacagtttccacgagagcacagaagggaaggtgggctacgaactgaaggatgagatcgagcgcaaattcgacaagtggcaggagccgccgcctgtgaagcaggtgaagccgctgcctgcgcccctggatggacagcggaagaagcgaggcggccgcaggtaccgcaagatgaaggagcggctggggctgacggagatccggaagcaggccaaccgtatgagcttcggagagatcgaggaggacgcctaccaggaggacctgggattcagcctgggccacctgggcaagtcgggcagtgggcgtgtgcggcagacacaggtaaacgaggccaccaaggccaggatctccaagacgctgcagcggaccctgcagaagcagagcgtcgtatatggcgggaagtccaccatccgcgaccgctcctcgggcacggcctccagcgtggccttcaccccactccagggcctggagattgtgaacccacaggcggcagagaagaaggtggctgaggccaaccagaagtatttctccagcatggctgagttcctcaaggtcaagggcgagaagagtggccttatgtccacctgaatgactgcgtgtgtccaaggtggcttcccactgaagggacacagaggtccagtccttctgaagggctaggatcgggttctggcagggagaacctgccctgccactggccccattgctgggactgcccagggaggaggccttggaagagtccggcctggcctcccccaggaccgagatcaccgcccagtatgggctagagcaggtcttcatcatgccttgtcttttttaactgagaaaggagattttttgaaaagagtacaattaaaaggacattgtcaaga

SEQ ID NO: 5 - protein, 499 aa, Homo sapiens
MSLADELLADLEEAAEEEEGGSYGEEEEEPAIEDVQEETQLDLSGDSVKTIAKLWDSKMFAEIMMKIEEYISKQAKASEVMGPVEAAPEYRVIVDANNLTVEIENELNIIHKFIRDKYSKRFPELESLVPNALDYIRTVKELGNSLDKCKNNENLQQILTNATIMVVSVTASTTQGQQLSEEELERLEEACDMALELNASKHRIYEYVESRMSFIAPNLSIIIGASTAAKIMGVAGGLTNLSKMPACNIMLLGAQRKTLSGFSSTSVLPHTGYIYHSDIVQSLPPDLRRKAARLVAAKCTLAARVDSFHESTEGKVGYELKDEIERKFDKWQEPPPVKQVKPLPAPLDGQRKKRGGRRYRKMKERLGLTEIRKQANRMSFGEIEEDAYQEDLGFSLGHLGKSGSGRVRQTQVNEATKARISKTLQRTLQKQSVVYGGKSTIRDRSSGTASSVAFTPLQGLEIVNPQAAEKKVAEANQKYFSSMAEFLKVKGEKSGLMST

SEQ ID NO: 6 - DNA, 22 bp, Artificial Sequence
  Description of Artificial Sequence Synthetic primer
gtcaggagatcgagaccatccc

SEQ ID NO: 7 - DNA, 20 bp, Artificial Sequence
  Description of Artificial Sequence Synthetic primer
tcctgcctcagcctcccaag

(19) United States
(12) Patent Application Publication
(10) Pub. No.: US 2022/0152278 A1
(43) Pub. Date: May 19, 2022

(54) IMPROVED SURVIVAL OF HUMAN CELLS DIFFERENTIATED IN VITRO BY PRPF31 GENE EXPRESSION KNOCKDOWN

(71) Applicant: UNIVERSITY OF WASHINGTON, Seattle, WA (US)

(72) Inventors: Charles E. MURRY, Seattle, WA (US); Sarah DUPRAS, Seattle, WA (US); John LAMACCHIA, Seattle, WA (US); James FUGATE, Seattle, WA (US); William Robb MACLELLAN, Seattle, WA (US); Lil PABON, Jr., Seattle, WA (US); Scott THIES, Seattle, WA (US); Hiroshi TSUCHIDA, Seattle, WA (US); Stephanie A. TUCK, Seattle, WA (US)

(73) Assignee: UNIVERSITY OF WASHINGTON, Seattle, WA (US)

(21) Appl. No.: 17/439,482

(22) PCT Filed: Mar. 13, 2020
(86) PCT No.: PCT/US2020/022679

§ 371 Date: Sep. 15, 2021

Related U.S. Application Data
(60) Provisional application No. 62/818,979, filed on Mar. 15, 2019.

(51) Int. Cl. A61L 27/38; A61K 31/7105; A61K 31/713; A61K 35/545; A61K 45/06; C12N 5/077

(52) U.S. Cl. CPC A61L 27/3804; A61K 31/7105; A61K 31/713; C12N 2506/45; A61K 45/06; C12N 5/0657; C12N 2501/65; A61K 35/545

(57) ABSTRACT

Described herein are methods and compositions related to methods of improving survival and engraftment of human cells differentiated in vitro, and uses thereof.

### CROSS-REFERENCE TO RELATED APPLICATION

This application claims benefit under 35 U.S.C. § 119(e) of U.S. Provisional Application No. 62/818,979 filed Mar. 15, 2019, the contents of which are incorporated herein by reference in its entirety.

### SEQUENCE LISTING

The instant application contains a Sequence Listing which has been submitted electronically in ASCII format and is hereby incorporated by reference in its entirety. Said ASCII copy, created on Mar. 13, 2020, is named 034186-094370WOPT_SL.txt and is 8,354 bytes in size.

### TECHNICAL FIELD

The technology described herein relates to methods of improving survival and engraftment of human cells differentiated in vitro, and uses thereof.

### BACKGROUND

At the turn of the millennium, cardiovascular disease has become widely identified as an emerging epidemic. Despite major advances with the treatment of heart failure and myocardial infarctions, human cell therapeutic approaches have fallen short of expected outcomes to repair cardiac tissues. This is due to the lack of survival of stem cell-derived cardiomyocytes following transplantation and their lack of stability in vivo. Therefore, new approaches to improve survival of human cells differentiated in vitro are needed to improve treatment outcomes for patients with cardiovascular disease, cardiac injuries, or other diseases that rely on stem cell or cell transplant therapies.

### SUMMARY

The methods and compositions described herein are related, in part, to the discovery that decreasing the level of Pre-mRNA Processing Factor 31 enhances the survival and/or engraftment of in vitro-differentiated cells.

In one aspect, described herein is a composition comprising human cells differentiated in vitro from stem cells and an agent that decreases the level or activity of Pre-mRNA Processing Factor 31 (PRPF31).

In one embodiment of any of the aspects, the composition is a transplant composition.

In another embodiment, the cells differentiated in vitro from stem cells are cardiomyocytes.

In another embodiment, the cells differentiated in vitro from stem cells are of a mesodermal lineage.

In another embodiment, the in vitro-differentiated cells are of a cell type selected from: cardiomyocytes, skeletal muscle cells, smooth muscle cells, kidney cells, endothelial cells, skin cells, adrenal cortex cells, bone cells, white blood cells, and microglial cells.

In another embodiment, the in vitro-differentiated human cells are differentiated from induced pluripotent stem cells (iPSCs) or from embryonic stem cells.

In another embodiment, the stem cells are derived from a healthy subject.

In another embodiment, the agent is a small molecule, a polypeptide, a nucleic acid molecule or a vector comprising a nucleic acid molecule.

In another embodiment, the agent comprises or encodes a nucleic acid molecule comprising an antisense sequence, an aptamer or an RNA interference molecule (RNAi) that targets PRPF31 or its RNA transcript.

In another embodiment, the vector is selected from the group consisting of: a plasmid and a viral vector.

In another embodiment, the RNAi molecule comprises the nucleic acid sequence of SEQ ID NO: 1.

In another aspect, described herein is a transplant composition for transplant to a recipient, the composition comprising in vitro-differentiated human mesodermal lineage cells that have been contacted with an agent that decreases the level or activity of PRPF31. In one embodiment of any of the aspects, the human mesodermal lineage cells are cardiomyocytes.

In another embodiment, the agent is selected from a small molecule, a polypeptide, a nucleic acid molecule or a vector comprising a nucleic acid molecule.

In another embodiment, the agent comprises or encodes a nucleic acid molecule comprising an antisense sequence, an aptamer or an RNA interference molecule (RNAi) that targets PRPF31 or its RNA transcript.

In another embodiment, the vector is selected from the group consisting of: a plasmid and a viral vector.

In another embodiment, the RNAi molecule comprises the nucleic acid sequence of SEQ ID NO: 1.

In another embodiment, the in vitro-differentiated human mesodermal lineage cells are differentiated from induced pluripotent stem cells (iPSCs) or from embryonic stem cells.

In another embodiment, the mesodermal lineage cells are differentiated from iPSCs derived from the transplant recipient.

In another aspect, described herein is a method of transplanting in vitro-differentiated human mesodermal lineage cells, the method comprising transplanting into or onto a tissue or organ of a subject in vitro-differentiated human mesodermal lineage cells that have been contacted with an agent that decreases the level or activity of PRPF31. In one embodiment of any of the aspects, the cells are cardiomyocytes.

In another embodiment, the contacted cells survive transplanting to a greater extent than cells not contacted with the agent.

In another embodiment, the cells are cardiomyocytes and the subject has suffered a cardiac infarction.

In another embodiment, the agent is a small molecule, a polypeptide, a nucleic acid molecule or a vector comprising a nucleic acid molecule.

In another embodiment, the agent comprises or encodes a nucleic acid molecule comprising an antisense sequence, an aptamer or an RNA interference molecule (RNAi) that targets PRPF31 or its RNA transcript.

In another embodiment, the vector is selected from the group consisting of: a plasmid and a viral vector.

In another embodiment, the RNAi molecule comprises the nucleic acid sequence of SEQ ID NO: 1.

In another embodiment, the human cardiomyocytes are differentiated from induced pluripotent stem cells (iPSCs) or from embryonic stem cells.

In another embodiment, the iPSCs are derived from the subject.

In another embodiment, the iPSCs are derived from a healthy donor.

In another aspect, described herein is a method of promoting survival and/or engraftment of transplanted human, in vitro-differentiated cardiomyocytes, the method comprising contacting human, in vitro-differentiated cardiomyocytes with an agent that decreases the level or activity of PRPF31, and transplanting the cells into cardiac tissue of a human subject in need thereof.

In one embodiment, the subject has suffered a cardiac infarct.

In another embodiment, the agent is a small molecule, a polypeptide, a nucleic acid molecule or a vector comprising a nucleic acid molecule.

In another embodiment, the agent comprises or encodes a nucleic acid molecule comprising an antisense sequence, an aptamer or an RNA interference molecule (RNAi) that targets PRPF31 or its RNA transcript.

In another embodiment, the vector is selected from the group consisting of: a plasmid and a viral vector.

In another embodiment, the RNAi molecule comprises the nucleic acid sequence of SEQ ID NO: 1.

In another aspect, described herein is a method of promoting survival and/or engraftment of transplanted mesoderm lineage cells, the method comprising: administering to a subject in need thereof mesoderm lineage cells contacted or treated with an agent that decreases the level or activity of PRPF31 in the subject.

In one embodiment, the mesoderm-derived cells are in vitro differentiated mesoderm lineage cells.

In another embodiment, the mesoderm lineage cells are differentiated in vitro from iPS cells or embryonic stem cells.

In another embodiment, the agent is a small molecule, a polypeptide, a nucleic acid molecule or a vector comprising a nucleic acid molecule.

In another embodiment, the agent comprises or encodes a nucleic acid molecule comprising an antisense sequence, an aptamer or an RNA interference molecule (RNAi) that targets PRPF31 or its RNA transcript.

In another embodiment, the vector is selected from the group consisting of: a plasmid and a viral vector.

In another embodiment, the RNAi molecule comprises the nucleic acid sequence of SEQ ID NO: 1.

In another embodiment, the iPSCs are derived from the subject.

In another embodiment, the iPSCs are derived from a healthy donor.

In another embodiment, the transplanted mesoderm lineage cells are of a cell type selected from: cardiomyocytes, skeletal muscle cells, smooth muscle cells, kidney cells, endothelial cells, skin cells, adrenal cortex cells, bone cells, white blood cells, and microglial cells.

### Definitions

For convenience, the meanings of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in cellular and molecular biology, and biochemistry can be found in The Merck Manual of Diagnosis and Therapy, 20th Edition, published by Merck Sharp & Dohme Corp., 2018 (ISBN 9780911910421, 0911910425); Robert S. Porter et al. (eds.), The Encyclopedia of Molecular Cell Biology and Molecular Medicine, published by Blackwell Science Ltd., 2008 (ISBN 3527305424, 9783527305421); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2016 (ISBN 9780815345510, 0815345518); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Laboratory Methods in Enzymology: RNA, Jon Lorsch (ed.) Elsevier, 2013 (ISBN: 9780124200371, 0124200370); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), Immunological Methods, Ivan Lefkovits, Benvenuto Pernis, (eds.) Elsevier Science, 2014 (ISBN: 9781483269993, 148326999X), the contents of which are all incorporated by reference herein in their entireties.

As used herein a “transplant composition” refers to a composition comprising an in vitro-differentiated cell or a population thereof. The composition can be formulated for administration to a subject as a transplant. Transplant compositions will comprise a pharmaceutically acceptable carrier, and can optionally comprise a matrix or scaffold for the cells. A transplant composition can be formulated for administration by injection or, for example, by surgical implantation.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment of any of the aspects, the subject is a mammal. In another embodiment of any of the aspects, the subject is human. In another embodiment, of any of the aspects, the subject is an experimental animal or animal substitute as a disease model. In another embodiment, of any of the aspects, the subject is a domesticated animal including companion animals (e.g., dogs, cats, rats, pigs, guinea pigs, hamsters etc.). A subject can have previously received a treatment for a disease, or have never received treatment for a disease. A subject can have previously been diagnosed with having a disease, or have never been diagnosed with a disease.

The term “healthy subject” as used herein refes to a subject that, at a minimum, lacks markers or symptoms of the disease or disorder to be treated.

As used herein the term “human stem cell” refers to a human cell that can self-renew and differentiate to at least one different cell type. The term “human stem cell” encompasses human stem cell lines, human-derived induced pluripotent stem (iPS) cells, human embryonic stem cells, human pluripotent stem cells, human multipotent stem cells, amniotic stem cells, placental stem cells, or human adult stem cells. In one embodiment of any of the aspects, the human stem cell is not derived from a human embryo.

The term “derived from,” used in reference to a stem cell means the stem cell was generated by reprogramming of a differentiated cell to a stem cell phenotype. The term “derived from,” used in reference to a differentiated cell means the cell is the result of differentiation, e.g., in vitro-differentiation, of a stem cell. As one example, “iPSC-CMs” or “induced pluripotent stem cell-derived cardiomyocytes” are used interchangeably to refer to cardiomyocytes derived from an induced pluripotent stem cell by in vitro differentiation of the stem cell.

As used herein, “in vitro-differentiated cells” refers to cells that are generated in culture, typically via step-wise differentiation from a precursor cell such as a human embryonic stem cell, an induced pluripotent stem cell, an early mesodermal, ectodermal, or endodermal cell, or a progenitor cell. Thus, for example, “in vitro-differentiated cardiomyocytes” are cardiomyocytes that are generated in culture, typically via step-wise differentiation from a precursor cell such as a human embryonic stem cell, an induced pluripotent stem cell, an early mesoderm cell, a lateral plate mesoderm cell or a cardiac progenitor cell.

The term “agent” refers to any entity to be administered to or contacted with a cell, tissue, organ or subject which is normally not present or not present at the levels being administered to the cell, tissue, organ, or subject. Agents can be selected from a group comprising: chemicals; small molecules; nucleic acids; nucleic acid analogues; proteins; peptides; peptidomimetics; peptide derivatives; peptide analogs; aptamers; antibodies; intrabodies; biological macromolecules; or functional fragments thereof. A nucleic acid can be RNA or DNA, and can be single or double stranded, and can include, for example, nucleic acids encoding a protein of interest, as well as nucleic acids such as RNA interference or small interfering RNA molecules, antisense RNA molecules, or aptamers that inhibit gene expression or protein function. Nucleic acids can include oligonucleotides, as well as nucleic acid analogues, for example, peptide-nucleic acid (PNA), pseudo-complementary PNA (pc-PNA), and locked nucleic acid (LNA), etc.

Nucleic acids can include sequence encoding proteins, for example, that act as transcriptional repressors, as well as sequence encoding antisense molecules, ribozymes, small inhibitory nucleic acids, for example, but not limited to RNAi, shRNAi, siRNA, micro RNAi (mRNAi), antisense oligonucleotides, etc. A protein and/or peptide or fragment thereof can be any protein of interest, for example, but not limited to; mutated proteins, therapeutic proteins, or truncated proteins, including, e.g., dominant negative mutant proteins, wherein the protein is normally absent or expressed at lower levels in the cell. Proteins can also include mutated proteins, genetically engineered proteins, recombinant proteins, chimeric proteins, antibodies, midibodies, tribodies, humanized proteins, humanized antibodies, chimeric antibodies, modified proteins and fragments thereof. An agent can be applied or introduced to cell culture medium, where it contacts the cell and induces its effects. Alternatively, an agent can be intracellular as a result of introduction of a nucleic acid encoding the agent into the cell and its transcription resulting in the production of the nucleic acid and/or protein agent within the cell. In some embodiments, the agent is any chemical, entity or moiety, including without limitation synthetic and naturally-occurring non-proteinaceous entities. In certain embodiments the agent is a small molecule. Small molecules can include chemical moieties including unsubstituted or substituted alkyl, aromatic, or heterocyclyl moieties including macrolides, leptomycins and related natural products or analogues thereof. In some embodiments, agents can be extracts made from biological materials such as bacteria, plants, fungi, or animal cells or tissues. In some embodiments, agents can be naturally occurring or synthetic compositions or functional fragments thereof. Agents can be known to have a desired activity and/or property, or can be selected from a library of diverse compounds.

The phrase “pharmaceutically acceptable” is employed herein to refer to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “substrate” refers to a structure, comprising a biocompatible material that provides a surface suitable for adherence and proliferation of cells. A nanopatterned substrate can further provide mechanical stability and support and can, for example, promote maturation of in vitro-differentiated cells, such as in vitro-differentiated muscle cells or in vitro-differentiated cardiomyocytes. A substrate, including but not necessarily limited to a nanopatterned substrate, can be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

As used herein, “administering” is used in the context of the placement of an agent (e.g. a small molecule) described herein, on or into a cell, tissue, organ or a subject, by a method or route which results in at least partial localization of the agent at a desired site, e.g., in vitro differentiated cells, the heart, kidney, blood, skin, or a region thereof, such that a desired effect(s) is produced (e.g., decreased PRPF31 level or activity). The agent described herein can be administered by any appropriate route which results in delivery to a desired location in the subject. The half-life of the agent after administration to a subject can be as short as a few minutes, hours, or days, e.g., twenty-four hours, to a few days, to as long as several years, i.e., long-term. “Administering” can also refer to the placement of in vitro differentiated cells, treated with an agent as described herein, into a tissue, organ or subject. In this context, “administering” is equivalent to “transplanting.”

As used herein, the term “transplanting” is used in the context of the placement of cells, e.g. in vitro-differentiated cells as described herein, into a subject, by a method or route which results in at least partial localization of the introduced cells at a desired site, such as a site of injury or repair, such that a desired effect(s) is produced. In some embodiments, the cells, e.g., cardiomyocytes, can be implanted or injected directly into or on the organ, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years or more, i.e., long-term engraftment. As one of skill in the art will appreciate, long-term engraftment of the in vitro-differentiated cells is desired, as many mature adult cells (e.g., cardiomyocytes) do not proliferate to an extent that the organ (e.g., heart) can heal from an acute injury involving cell death.

A “treatment” of a disorder or a disease, (e.g., a cardiovascular disease) as referred to herein refers to therapeutic intervention that enhances the function of a cell, tissue, or organ, and/or enhances engraftment, and/or enhances transplant or graft vascularization in a treated area, thus improving the function of the tissue or organ, as non-limiting example, the heart. That is, a “treatment” is oriented to the function of the tissue or organ being treated (e.g., enhanced function within an infarcted area of the heart), and/or other site treated with the compositions described herein. Effective treatment need not cure or directly impact the underlying cause of the disease or disorder to be considered effective treatment. For example, a therapeutic approach that improves the function of the heart, e.g., in terms of contractile strength, or rhythm can be effective treatment without necessarily treating the cause of an infarction or arrhythmia.

As used herein, the terms “disease” or “disorder” refers to a disease, syndrome, or disorder, partially or completely, directly or indirectly, caused by one or more abnormalities in the genome, physiology, behavior, or health of a subject.

The disease or disorder can be a cardiac disease or disorder. Non-limiting examples of cardiac diseases include cardiomyopathy, cardiac arrhythmia, heart failure, arrhythmogenic right ventricular dysplasia (ARVD), long QT syndrome, catecholaminergic polymorphic ventricular tachycardia (CPVT), Barth syndrome, and cardiac involvement in Duchenne muscular dystrophy.

As used herein, “prevention” or “preventing,” when used in reference to a disease, disorder or symptoms thereof, refers to a reduction in the likelihood that an individual will develop a disease or disorder, e.g., heart failure following myocardial infarction, as but one example. The likelihood of developing a disease or disorder is reduced, for example, when an individual having one or more risk factors for a disease or disorder either fails to develop the disorder or develops such disease or disorder at a later time or with less severity, statistically speaking, relative to a population having the same risk factors and not receiving treatment as described herein. The failure to develop symptoms of a disease, or the development of reduced (e.g., by at least 10% on a clinically accepted scale for that disease or disorder) or delayed (e.g., by days, weeks, months or years) symptoms is considered effective prevention.

The terms “decrease”, “reduced”, “reduction”, “to a lesser extent,” or “inhibit” are all used herein to mean a decrease or lessening of a property, level, or other parameter by a statistically significant amount. In some embodiments, “reduced,” “reduction,” “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “increases,” or “enhance” or “activate” or “to a greater extent” are all used herein to generally mean an increase of a property, level, or other parameter by a statistically significant amount; for the avoidance of any doubt, the terms “increased”, “increase,” “to a greater extent,” “enhance” or “activate” can refere to an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, a “reference level” refers to the level of a marker or parameter in a normal, otherwise unaffected cell population or tissue (e.g., a cell, tissue, or biological sample obtained from a healthy subject, or a biological sample obtained from the subject at a prior time point, e.g., cell, tissue, or a biological sample obtained from a patient prior to being diagnosed with a disease, or a biological sample that has not been contacted with an agent or composition as disclosed herein). Alternatively, a reference level can also refer to the level of a given marker or parameter in a subject, organ, tissue, or cell, prior to administration of a treatment, e.g., with an agent or via administration of a transplant composition.

As used herein, an “appropriate control” refers to an untreated, otherwise identical cell, subject, organism, or population (e.g., a cell, tissue, or biological sample that was not contacted by an agent or composition described herein) relative to a cell, tissue, biological sample, or population contacted or treated with a given treatment. For example, an appropriate control can be a cell, tissue, organ or subject that has not been contacted with an agent or administered a cell as described herein.

The term “statistically significant” or “significantly” refers to statistical significance and generally means a two standard deviation (2SD) or greater difference.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

### BRIEF DESCRIPTION OF THE DRAWINGS

FIG. 1 demonstrates gene knockdown in a hPSC-CM derived from the RUES2 embryonic stem cell line. The hPSC-CMs were transfected with 5 nM siRNA using Lipofectamine RNAiMax (Thermo Fisher) incubation for 48 hours. Controls were untreated or transfected with a negative control scrambled siRNA. The efficiency of knockdown was confirmed by quantitative rtPCR. The resultant cells were cryopreserved for transplantation.

FIG. 2 demonstrates that survival of hPSC-CM with PRPF31 knockdown was increased compared to untreated and control siRNA-treated hPSC-CM (p=0.008 and p=0.007, respectively; unpaired t test).

### DETAILED DESCRIPTION

The compositions and methods described herein are related, in part, to the discovery that human pluripotent stem cell-derived cells of mesodermal lineage treated to decrease the level or activity of Pre-mRNA Processing Factor (PRPF31) survive better than untreated cells when transplanted to a tissue, organ or subject. In particular, it was found that human pluripotent stem cell-derived cardiomyocytes (hPSC-CM) survive and/or engraft in cardiac tissue with increased efficiency following transplant to such tissue.

Thus, described herein are methods of promoting survival and/or engraftment of transplanted mesoderm lineage cells, the method comprising: administering to a subject in need thereof mesoderm lineage cells that have been treated with an agent that decreases the level or activity of PRPF31.

In certain embodiments, the cells are in vitro-differentiated cells, including but not limited to in vitro differentiated cardiomyocytes, among others. In addition to methods for transplanting cells and for promoting survival of such cells, the technology described herein includes compositions comprising cells treated with an agent that decreases levels or activity of PRPF31 and cells in admixture with such an agent.

The following describes considerations relevant to the practice of the technology described.

Cell Preparations:

In certain embodiments, the compositions and methods described herein use in vitro-differentiated cells. Such cells can be differentiated from induced pluripotent stem cells (iPSCs) or from embryonic stem cells.

The following describes various sources and stem cells that can be used to prepare cells for transplant or engraftment into a subject.

Stem cells are cells that retain the ability to renew themselves through mitotic cell division and can differentiate into more specialized cell types. Three broad types of mammalian stem cells include: embryonic stem (ES) cells that are found in blastocysts, induced pluripotent stem cells (iPSCs) that are reprogrammed from somatic cells, and adult stem cells that are found in adult tissues. Other sources of stem cells can include, for example, amnion-derived or placental-derived stem cells. Pluripotent stem cells can differentiate into cells derived from any of the three germ layers.

Cells useful in the compositions and methods described herein can be differentiated from both embryonic stem cells and induced pluripotent stem cells, among others.

In one embodiment, the compositions and methods provided herein use mesodermal lineage cells, including but not limited to human cardiomyocytes differentiated from embryonic stem cells. Alternatively, in some embodiments, the compositions and methods provided herein do not encompass generation or use of differentiated human cells derived from cells taken from a viable human embryo.

Embryonic stem cells: Embryonic stem cells and methods for their retrieval are described, for example, in Trounson A. O. Reprod. Fertil. Dev. (2001) 13: 523, Roach M L Methods Mol. Biol. (2002) 185: 1, and Smith A. G. Annu Rev Cell Dev Biol (2001) 17:435. The term “embryonic stem cell” is used to refer to the pluripotent stem cells of the inner cell mass of the embryonic blastocyst (see e.g., U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). Markers of embryonic stem cells include, for example, any one or any combination of Oct3, Nanog, SOX2, SSEA1, SSEA4 and TRA-1-60.

Cells derived from embryonic sources can include embryonic stem cells or stem cell lines obtained from a stem cell bank or other recognized depository institution. Other means of producing stem cell lines include methods comprising the use of a blastomere cell from an early stage embryo prior to formation of the blastocyst (at around the 8-cell stage). Such techniques use, for example, single cells removed in the pre-implantation genetic diagnosis technique routinely practiced in assisted reproduction clinics. The single blastomere cell is co-cultured with established ES-cell lines and then separated from them to form fully competent ES cell lines.

Undifferentiated embryonic stem (ES) cells are easily recognized by those skilled in the art, and typically appear in the two dimensions of a microscopic view as colonies of cells with high nuclear/cytoplasmic ratios and prominent nucleoli. Markers of embryonic stem cells include, for example, any one or any combination of Oct3, Nanog, SOX2, SSEA1, SSEA4 and TRA-1-60. In some embodiments, the differentiated human cells for use in the methods and compositions described herein are not derived from embryonic stem cells or any other cells of embryonic origin.

Induced Pluripotent Stem Cells (iPSCs): In some embodiments, the compositions and methods described herein utilize human cardiomyocytes or other human mesodermal lineage cells that are differentiated in vitro from induced pluripotent stem cells. An advantage of using iPSCs to generate cells for the compositions and methods described herein is that, if so desired, the cells can be derived from the same subject to which the differentiated cells are to be administered. That is, a somatic cell can be obtained from a subject, reprogrammed to an induced pluripotent stem cell, and then re-differentiated into a human cardiomyocyte or other mesodermal lineage cell to be administered to the subject (i.e., autologous cells). Since the cells and their differentiated progeny are essentially derived from an autologous source, the risk of engraftment rejection or allergic responses is reduced compared to the use of cells from another subject or group of subjects. While this is an advantage of iPS cells, in alternative embodiments, the cardiomyocytes and other human mesodermal lineage cells useful for the methods and compositions described herein are derived from non-autologous sources (i.e., allogenic cells). In addition, the use of iPSCs negates the need for cells obtained from an embryonic source.

Although differentiation is generally irreversible under physiological contexts, several methods have been developed in recent years to reprogram somatic cells to induced pluripotent stem cells. Exemplary methods are known to those of skill in the art and are described briefly herein below.

Reprogramming is a process that alters or reverses the differentiation state of a differentiated cell (e.g., a somatic cell). Stated another way, reprogramming is a process of driving the differentiation of a cell backwards to a more undifferentiated or more primitive type of cell. It should be noted that placing many primary cells in culture can lead to some loss of fully differentiated characteristics. However, simply culturing such cells included in the term differentiated cells does not render these cells non-differentiated cells or pluripotent cells. The transition of a differentiated cell to pluripotency requires a reprogramming stimulus beyond the stimuli that lead to partial loss of differentiated character when differentiated cells are placed in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. Thus, cells to be reprogrammed can be terminally differentiated somatic cells, as well as adult or somatic stem cells.

In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell to an undifferentiated cell (e.g., an embryonic-like cell). Reprogramming can result in expression of particular genes by the cells, the expression of which further contributes to reprogramming. In certain embodiments described herein, reprogramming of a differentiated cell causes the differentiated cell to assume an undifferentiated state with the capacity for self-renewal and differentiation to cells of all three germ layer lineages. These are induced pluripotent stem cells (iPSCs or iPS cells).

Methods of reprogramming somatic cells into iPS cells are described, for example, in U.S. Pat. Nos. 8,129,187 B2; 8,058,065 B2; US Patent Application 2012/0021519 A1; Singh et al. Front. Cell Dev. Biol. (February, 2015); and Park et al., Nature 451: 141-146 (2008); which are incorporated by reference in their entireties. Specifically, iPSCs are generated from somatic cells by introducing a combination of reprogramming transcription factors. The reprogramming factors can be introduced as, for example, proteins, nucleic acids (mRNA molecules, DNA constructs or vectors encoding them) or any combination thereof. Small molecules can also augment or supplement introduced transcription factors. While additional factors have been determined to affect, for example, the efficiency of reprogramming, a standard set of four reprogramming factors sufficient in combination to reprogram somatic cells to an induced pluripotent state includes Oct4 (Octamer binding transcription factor-4), SOX2 (Sex determining region Y)-box 2, Klf4 (Kruppel Like Factor-4), and c-Myc. Additional protein or nucleic acid factors (or constructs encoding them) including, but not limited to LIN28+Nanog, Esrrb, Pax5 shRNA, C/EBPa, p53 siRNA, UTF1, DNMT shRNA, Wnt3a, SV40 LT(T), hTERT) or small molecule chemical agents including, but not limited to BIX-01294, BayK8644, RG108, AZA, dexamethasone, VPA, TSA, SAHA, PD0325901+CHIR99021(2i) and A-83-01 have been found to replace one or the other reprogramming factors from the basal or standard set of four reprogramming factors, or to enhance the efficiency of reprogramming.

The specific approach or method used to generate pluripotent stem cells from somatic cells (e.g., any cell of the body with the exclusion of a germ line cell; fibroblasts, etc.) is not critical to the claimed invention. Thus, any method that re-programs a somatic cell to the pluripotent phenotype would be appropriate for use in the methods described herein.

The efficiency of reprogramming (i.e., the number of reprogrammed cells) derived from a population of starting cells can be enhanced by the addition of various small molecules as shown by Shi, Y., et al. (2008) Cell-Stem Cell 2:525-528, Huangfu, D., et al. (2008) Nature Biotechnology 26(7):795-797, and Marson, A., et al. (2008) Cell-Stem Cell 3:132-135. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

To confirm the induction of pluripotent stem cells for use with the methods described herein, isolated clones can be tested for the expression of one or more stem cell markers. Such expression in a cell derived from a somatic cell identifies the cells as induced pluripotent stem cells. Stem cell markers can include but are not limited to SSEA3, SSEA4, CD9, Nanog, Oct4, Fbx15, Ecat1, Esg1, Eras, Gdf3, Fgf4, Cripto, Daxl, Zpf296, Slc2a3, Rex1, Utf1, and Nat1, among others. In one embodiment, a cell that expresses Nanog and SSEA4 is identified as pluripotent. Methods for detecting the expression of such markers can include, for example, RT-PCR and immunological methods that detect the presence of the encoded polypeptides, such as Western blots or flow cytometric analyses. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry using antibodies specific for markers of the different germ line lineages is performed on a tumor arising from the cells. The growth of a tumor comprising cells from all three germ layers, endoderm, mesoderm and ectoderm further indicates or confirms that the cells are pluripotent stem cells.

Adult Stem Cells: Adult stem cells are stem cells derived from tissues of a post-natal or post-neonatal organism or from an adult organism. An adult stem cell is structurally distinct from an embryonic stem cell not only in markers it does or does not express relative to an embryonic stem cell, but also by the presence of epigenetic differences, e.g. differences in DNA methylation patterns. It is contemplated that cardiomyocytes and/or neurons differentiated from adult stem cells can also be used for the methods described herein. Methods of isolating adult stem cell are described for example, in U.S. Pat. No. 9,206,393 B2; and US Application No. 2010/0166714 A1; which are incorporated herein by reference in their entireties.

In Vitro-Differentiation

Certain methods and compositions as described herein use moesodermal lineage cells differentiated in vitro from stem cells. Generally, throughout the differentiation process, a pluripotent cell will follow a developmental pathway along a particular developmental lineage, e.g., the primary germ layers-ectoderm, mesoderm, or endoderm.

The embryonic germ layers are the source from which all tissues and organs derive. The mesoderm is the source of, for example, smooth and striated muscle, including cardiac muscle, connective tissue, vessels, the cardiovascular system, blood cells, bone marrow, skeleton, reproductive organs and excretory organs.

The germ layers can be identified by the expression of specific biomarkers and gene expression. Assays to detect these biomarkers include, e.g., RT-PCR, immunohistochemistry, and Western blotting. Non-limiting examples of biomarkers expressed by early mesodermal cells include HAND1, ESM1, HAND2, HOPX, BMP10, FCN3, KDR, PDGFR-α, CD34, Tbx-6, Snail-1, Mesp-1, and GSC, among others. Biomarkers expressed by early ectoderm cells include but are not limited to TRPM8, POU4F1, OLFM3, WNT1, LMX1A and CDH9, among others. Biomarkers expressed by early endoderm cells include but are not limited to LEFTY1, EOMES, NODAL and FOXA2, among others. One of skill in the art can determine which lineage markers to monitor while performing a differentiation protocol based on the cell type and the germ layer from which that cell is derived in development.

Induction of a particular developmental lineage in vitro is accomplished by culturing stem cells in the presence of specific agents or combinations thereof that promote lineage commitment. Generally, the methods described herein comprise the step-wise addition of agents (e.g., small molecules, growth factors, cytokines, polypeptides, vectors, etc.) into the cell culture medium or contacting a cell with agents that promote differentiation. In particular, mesoderm formation is induced by transcription factors and growth factor signalling which includes but is not limited to VegT, Wnt signalling (e.g., via β-catenin), bone morphogenic protein (BMP) pathways, fibroblast growth factor (FGF) pathways, and TGFβ signalling (e.g., activin A). See e.g., Clemens et al. Cell Mol Life Sci. (2016), which is incorporated herein by reference in its entirety.

In the context of cell ontogeny, the term “differentiate”, or “differentiating” is a relative term meaning a “differentiated cell” is a cell that has progressed further down the developmental pathway than its precursor cell. Thus, in some embodiments, a reprogrammed cell can differentiate to lineage-restricted precursor cells (such as a mesodermal stem cell), which in turn can differentiate into other types of precursor cells further down the pathway (such as a tissue specific precursor, e.g., a cardiomyocyte precursor), and then to an end-stage differentiated cell, which plays a characteristic role in a certain tissue type, and may or may not retain the capacity to proliferate further.

Generally, in vitro-differentiated cells will exhibit a down-regulation of pluripotency markers (e.g., HNF4-α, AFP, GATA-4, and GATA-6) throughout the step-wise process and exhibit an increase in expression of lineage-specific biomarkers (e.g., mesodermal, ectodermal, or endodermal markers). See for example, Tsankov et al. Nature Biotech (2015), which describes the characterization of human pluripotent stem cell lines and differentiation along a particular lineage. The differentiation process can be monitored for efficiency by a number of methods known in the art. This includes detecting the presence of germ layer biomarkers using standard techniques, e.g., immunocytochemistry, RT-PCR, flow cytometry, functional assays, optical tracking, etc.

In some embodiments of any of the aspects, the in vitro-differentiated cells are of a mesodermal lineage cell type selected from: cardiomyocytes, skeletal muscle cells, smooth muscle cells, kidney cells, liver cells, endothelial cells, skin cells, adrenal cortex cells, bone cells, white blood cells, and microglial cells.

Cardiomyocyte Differentiation:

In some embodiments of the methods and compositions described herein, the cells differentiated in vitro from stem cells are cardiomyocytes. Methods for the differentiation of cardiomyocytes from ESCs or iPSCs are known in the art. In some embodiments of any of the aspects, the cardiomyocytes are differentiated from iPSCs derived from the transplant recipient, e.g., as described herein or as known in the art.

In certain embodiments, the step-wise differentiation of ESCs or iPSCs to cardiomyocytes proceeds in the following order: ESC or iPSC>cardiogenic mesoderm>cardiac progenitor cells>cardiomyocytes (see e.g., Lian et al. Nat Prot (2013); US Applicant No. 2017/0058263 A1; 2008/0089874 A1; 2006/0040389 A1; U.S. Pat. Nos. 10,155,927 B2; 9,994,812 B2; and 9,663,764 B2, the contents of each of which are incorporated herein by reference their entireties). See also, e.g., LaFlamme et al., Nature Biotech 25:1015-1024 (2007), which is incorporated herein by reference in its entirety. In these differentiation protocols, agents can be added or removed from cell culture media to direct differentiation to cardiomyocytes in a step-wise fashion. Non-limiting examples of factors and agents that can promote cardiomyocyte differentiation include small molecules (e.g., Wnt inhibitors, GSK3 inhibitors), polypeptides (e.g., growth factors), nucleic acids, vectors, and patterned substrates (e.g., nanopatterns). The addition of growth factors necessary in cardiovascular development, including but not limited to fibroblast growth factor 2 (FGF2), transforming growth factor β (TGFβ) superfamily growth factors Activin A and BMP4, vascular endothelial growth factor (VEGF), and the Wnt inhibitor DKK-1, can also be beneficial in directing differentiation along the cardiac lineage. Additional examples of factors and conditions that help promote cardiomyocyte differentiation include but are not limited to B27 supplement lacking insulin, cell-conditioned media, external electrical pacing, and nanopatterned substrates, among others.

By way of example only, embryonic stem cells or iPS cells can be cultured in embryonic fibroblast conditioned medium (e.g., mouse, MEF-CM) and seeded onto an extracellular matrix (e.g., Matrigel®, a gelatin protein mixture secreted by Engelbreth Holm-Swarm (EHS) mouse sarcoma cells). To begin to differentiate cardiomyocytes, cells are administered new medium with basic fibroblast growth factor (bFGF) for about 6-7 days. After 7 days, the fibroblast conditioned medium is replaced with a Roswell Park Memorial Institute 1640 Medium comprising B27 supplement (referred to herein as RPMI-B27) and supplemented with cytokines as follows: (a) treatment with 100 ng/ml human recombinant activin A for about 24 hours, followed by (b) treatment with 10 ng/ml human recombinant BMP4 for about 4 days. The medium can then be exchanged for RPMI-B27 medium without the supplementary cytokines and cultures are fed new medium every 2-3 days for 2-3 additional weeks.

Generally, cells being differentiated into cardiomyocytes will begin to beat and contract in culture about 12 days after the addition of activin A. This can be monitored using standard cell culture and microscopy techniques.

In addition to in vitro-differentiated cardiomyocyte functional readouts (e.g., beating cells), the in vitro-differentiated cardiomyocytes will also express biomarkers specific to adult cardiac cells. Non-limiting examples of cardiomyocyte biomarkers include cardiac troponin T (cTnT), α-actinin, or myosin heavy chain. While additional protein markers, and, e.g., functional hallmarks of cardiomyocyte maturity are preferred to be present, at a minimum in vitro-differentiated human cardiomyocytes useful in the methods and compositions described herein will express cardiac troponin T. If necessary or desired, the cardiomyocytes can then be enriched for using a Percoll gradient or a cell sorting technique (e.g., flow cytometry) for cardiomyocyte biomarkers (e.g., troponin T, α-actinin, myosin heavy chain, or ryanodine receptor 2). Examples of cardiomyocyte enrichment are found, e.g., in Xu et al. Circ Res. (2002); Laflamme et al. Am. J. Pathol. 167, 663-671 (2005); and Miltenyi Biotec MACS® Characterization by flow cytometry PSC-derived cardiomyocyte subtypes (2017); which are incorporated herein by reference in their entireties.

In vitro-differntiated cardiomyocyte maturity can be assessed by a number of parameters such as electrical maturity of a cell, metabolic maturity of a cell, or contractile maturity of an in vitro-differentiated cell. Examples of cardiomyocyte maturity proteins, biochemical, and electrical maturity markers are found, e.g., in WO2019/035032 A2, which is incorporated herein by reference in its entirety.

Non-limiting examples of such methods to determine electrical maturity of a cell include whole cell patch clamp (manual or automated), multielectrode arrays, field potential stimulation, calcium imaging and optical mapping, among others. Cells can be electrically stimulated during whole cell current clamp or field potential recordings to produce an electrical and/or contractile response. Measurement of field potentials and biopotentials of cardiomyocytes can be used to determine the differentiation stage and cell maturity.

With regard to cardiomyocytes, electrical maturity is determined by one or more of the following markers as compared to a reference level: increased gene expression of one or more ion channel genes, increased sodium current density, increased inwardly-rectifying potassium channel current density, increased action potential frequency, increased calcium wave frequency, and increased field potential frequency. Methods of measuring gene expression are known in the art, e.g., RT-PCR and transcriptomic sequencing.

Metabolic assays can be used to determine the differentiation stage and cell maturity of the in vitro-differentiated cells as described herein. Non-limiting examples of metabolic assays include cellular bioenergetics assays (e.g., Seahorse Bioscience XF Extracellular Flux Analyzer), and oxygen consumption tests. Specifically, cellular metabolism can be quantified by oxygen consumption rate (OCR), OCR trace during a fatty acid stress test, maximum change in OCR, maximum change in OCR after FCCP addition, and maximum respiratory capacity among other parameters. Furthermore, a metabolic challenge or lactate enrichment assay can provide a measure of cellular maturity or a measure of the effects of various treatments of such cells

For example, metabolic maturity of in vitro-differentiated cardiomyocytes is determined by one or more of the following markers as compared to a reference level: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA relative to immature in vitro-differentiated cardiomyocytes. Mammalian cells generally use glucose as their main energy source. However, cardiomyocytes are capable of energy production from different sources such as lactate or fatty acids. In some embodiments, lactate-supplemented and glucose-depleted culture medium, or the ability of cells to use lactate or fatty acids as an energy source is useful to identify mature cardiomyocytes and variations in their function.

Contractile maturity of an in vitro-differentiated cell (e.g, cardiomyocytes, skeletal muscle, or smooth muscle) is determined by one or more of the following markers as compared to a reference level: increased beat frequency, increased contractile force, increased level or activity of α-myosin heavy chain (α-MHC), increased level or activity of sarcomeres, decreased circularity index, increased level or activity of troponin, increased level or activity of titin N2b, increased cell area, and increased aspect ratio. Contractility can be measured by optical tracking methods such as video analysis. For video tracking methods, displacement of tissues or single cells can be measured to determine contractile force, frequency, etc.

Additional Cell Types:

The methods and compositions described herein also use or are applicable to in vitro-differentiated mesodermal lineage cells including, skeletal muscle cells, smooth muscle cells, kidney cells, endothelial cells, skin cells, adrenal cortex cells, bone cells, white blood cells, and microglial cells.

Methods of differentiating stem cell-derived skeletal muscle cells, smooth muscle, and/or adipose cells are described, e.g., in U.S. Pat. No. 10,240,123 B2; and Cheng et al. Am J Physiol Cell Physiol (2014). Methods of differentiating kidney cells are described, e.g., in Tajiri et al. Scientific Reports 8:14919 (2018); Taguchi et al. Cell Stem Cell 14:53-67 (2014); and US application 2010/0021438 A1. Methods of differentiating endothelial cells (e.g., vascular endothelium) are described in, e.g., U.S. Pat. No. 10,344,262 B2, and Olgasi et al., Stem Cell Reports 11:1391-1406 (2018). Methods of differentiating hormone-producing cells are described, e.g., in U.S. Pat. No. 7,879,603 B2, and Abu-Bonsrah et al. Stem Cell Reports 10:134-150 (2018). Methods of differentiating bone cells are described, e.g., in Csobonyeiova et al. J Adv Res 8: 321-327 (2017), U.S. Pat. Nos. 7,498,170 B2; 6,391,297 B1; and US application No. 2010/0015164 A1. Methods of differentiating microglial cells are described, e.g., in WO 2017/152081 A1. Methods of differentiating epithelial cells and skin cells are described, e.g., in Kim et al., Stem Cell Research and Therapy (2018); U.S. Pat. Nos. 7,794,742 B2; 6,902,881 B2. Methods of differentiating blood cells and white blood cells are described, e.g., in U.S. Pat. Nos. 6,010,696 A and 6,743,634 B2. Methods of differentiating stem cell-derived beta cells are described, e.g., in WO 2016/100930A1. Each of the above references are incorporated herein by reference in their entireties.

Methods of Enriching for Specific Cell Types:

The stem cells, progenitor cells, and/or in vitro-diffentiated cells described herein can be cultured on a mouse embryonic fibroblast (MEF) feeder layer of cells, Matrigel®, collagenase IV, or any other matrix or scaffold that substantially promotes in-vitro differentiation of the desired cell type and/or maintains a mature, viable, phenotype of the desired cell. In some embodiments, antibodies or similar agents specific for a given marker, or set of markers, can be used to separate and isolate the desired cells using fluorescent activated cell sorting (FACS), panning methods, magnetic particle selection, particle sorter selection and other methods known to persons skilled in the art, including density separation (Xu et al. (2002) Circ. Res. 91:501; U.S.S.N. 20030022367) and separation based on other physical properties (Doevendans et al. (2000) J. Mol. Cell. Cardiol. 32:839-851). Negative selection can be performed, including selecting and removing cells with undesired markers or characteristics, for example fibroblast markers, epithelial cell markers etc.

Undifferentiated ES cells express genes that can be used as markers to detect the presence of undifferentiated cells. Exemplary ES cell markers include stage-specific embryonic antigen (SSEA)-3, SSEA-4, TRA-I-60, TRA-1-81, alkaline phosphatase or those described in e.g., U.S.S.N. 2003/0224411; or Bhattacharya (2004) Blood 103(8):2956-64, each herein incorporated by reference in their entirety. Exemplary markers expressed on cardiac progenitor cells include, but are not limited to, TMEM88, GATA4, ISL1, MYL4, and NKX2-5. Such markers can be assessed or used to remove or determine the presence of undifferentiated or progenitor cells in, e.g., a population of in vitro-differentiated cardiomyocytes. Similarly, the presence of markers of undifferentiated cells, whether embryonic markers or otherwise, can be used to evaluate populations of other mesoderm lineage cell types useful in the methods and compositions described herein.

Agents that Reduce the Levels and/or Activity of PRPF31

Pre-mRNA Processing Factor 31, also called U4/U6 small nuclear ribonucleoprotein Prp31; hPRP31 or PRPF31, is a component of the splieceosome encoded by the gene PRPF31. PRPF31 is a ubiquitously expressed 61-kDa splicing factor protein that activates the spilceosome complex. The spliceosome complex is comprised of polypeptides and small nuclear RNAs (snRNAs) that function to remove introns, the non-coding regions of transcribed pre-RNAs, in the RNA splicing process. The addition of PRPF31 is neccessary for the transition of the spliceosomal complex to the activated state (see e.g., Liu et al., 2007, and Schaffert et al. EMBO J. (2014) which are incorporated herein by reference in their entireties).

The gene, mRNA and amino acid sequences of PRPF31 are known in the art, e.g., the human PRPF31 gene (NCBI GenelD: 26121)), the human mRNA transcript (NCBI Reference Sequence: NM_015629.4 (SEQ ID NO: 4)), and the human amino acid sequence (NCBI Reference Sequence: NP_056444.3 (SEQ ID NO: 5)).

In certain embodiments, methods and compositions described herein include the use of an agent or agents that inhibit or decrease the level or activity of PRPF31 in cells or cell preparations for transplant, e.g., in vitro-differentiated cells for transplant.

The levels of PRPF31 can be determined by methods known in the art, for example, immunoprecipitation or other pull down assays, western blotting, qPCR, RT-PCR, and immunocytochemistry. Thus, these methods can be used to determine whether a given treatment or agent decreases the level of PRPF31 protein, mRNA, or both. Primers for RT-PCR can be prepared on the basis of the mRNA sequence, e.g., based on SEQ ID NO: 5. Antibodies that specifically bind human PRPF31 are available, e.g., from Novus Biologicals® (Centennial, Colo.), Santa Cruz Biotechnology® (Dallas, Tex.), and Abcam® (Cambridge, Mass.) and can be used, e.g., to detect changes in PRPF31 following treatment with an agent that decreases the level of PRPF31 in e.g., in vitro-differentiated mesodermal lineage cells, such as cardiomyocytes, among others.

In some embodiments, an agent decreases the activity of PRPF31. In some embodiments the agent decreases the activity of PRPF31 by at least 20%, at least 30%, at least 40%, at least 50%, at least 60%, at least 70%, at least 80%, at least 90%, or more as compared to an appropriate control.

The activity of PRPF31 can be determined by any method known in the art. For example, the activity of PRPF31 in splicing can be assayed using a minigene constructed for a transfection-based assay as described by Wilke et al., Mol. Vis. 14:683-690 (2008), which is incorporated herein by reference in its entirety. While not wishing to be bound by theory, it is contemplated that the effect of PRPF31 inhibition on promotion of survival or engraftment of transplanted cells is related to PRPF31's activity in mRNA splicing. PRPF31 binds to U4 snRNP in the U4/U6 snRNP complex and is thought to form a bridge between the U4/U6 di-snRNP and U5 by binding to the U5 specific PRPF6 protein. See e.g., Makarova et al., EMBO J. 21:1148-1157 (2002). Thus, in another approach, one can evaluate PRPF31 activity by assaying its interaction with PRPF6, either in cells or in vitro, e.g., via co-immunoprecipitation or other assay for PRPF31/PRPF6 complex formation.

It is alternatively contemplated that the activity of PRPF31 in promoting survival and/or engraftment is not dependent upon the activity of the factor in splicing. Agents that, for example, bind to PRPF31 or promote modification of PRPF31 can be evaluatated for inhibition of PRPF31 activity.

In one embodiment, the effect of an agent that decreases PRPF31 activity can be confirmed by contacting in vitro-differentiated cells, e.g., cells of a mesodermal lineage, e.g., in vitro-differentiated cardiomyocytes, with the agent and transplanting the cells into an appropriate animal model. An agent that promotes survival of the transplanted cells relative to untreated cells is then confirmed to be an agent that decreases PRPF31 activity.

The Wilke et al. publication also describes a pull-down assay measuring this complex formation, as well as a mutant PRPF31 polypeptide, with an A216P missense mutation that acts in a dominant negative manner on splicing. It is contemplated that transient expression of the A216P mutant protein could be used to decrease PRPF31 activity in in vitro-differentiated cells used for transplant in methods and compositions as described herein.

In some embodiments of any of the aspects, the agent is a small molecule, a polypeptide, an antibody, a nucleic acid molecule, an RNAi, a vector comprising a nucleic acid molecule, an antisense oligonucleotide, or a gene editing system.

In some embodiments, an agent decreases the level of PRPF31. In some embodiments the agent decreases the level of PRPF31 by at least 20%, at least 30%, at least 40%, at leasat 50%, at least 60%, at least 70%, at least 80%, at least 90%, or more as compared to an appropriate control.

In some embodiments, the agent that decreases the level or activity of PRPF31 is a small molecule. A small molecule is an organic or inorganic molecule, which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, and salts, esters, and other pharmaceutically acceptable forms of such compounds. Examples of “small molecules” include, but are not limited to, compounds described in Goodman and Gillman's “The Pharmacological Basis of Therapeutics” 13 ed. (2018); incorporated herein by reference. Methods for screening small molecules are known in the art and can be used to identify a small molecule that is efficient at, for example, modulating PRPF31 levels or activity, given the desired target (e.g., PRPF31 polypeptide).

In some embodiments of any of the aspects, the agent that decreases the level or activity of PRPF31 comprises or encodes a nucleic acid molecule comprising an antisense sequence, an aptamer or an RNA interference molecule (RNAi) that targets PRPF31 or its RNA transcript.

In some embodiments, of any of the aspects, the inhibitory nucleic acid is an inhibitory RNA or RNA interference molecule (iRNA).

RNAi, also referred to as interfering RNA (iRNA) is any of a class of agents that contain RNA (or modified nucleic acids as described, for example, herein below) and which mediates the targeted cleavage of an RNA transcript via a highly conserved RNA-induced silencing complex (RISC) pathway. In some embodiments of any of the aspects, an iRNA as described herein effects inhibition of the expression and/or activity of a target, e.g. PRPF31. In some embodiments of any of the aspects, contacting a cell with the inhibitor (e.g. an iRNA) results in a decrease in the target mRNA level in a cell by at least about 10%, about 20%, about 30%, about 40%, about 50%, about 60%, about 70%, about 80%, about 90%, about 95%, about 99%, up to and including 100% of the target mRNA level found in the cell without the presence of the iRNA.

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target, e.g., it can span one or more intron boundaries. The other strand (the sense strand) includes a region that is complementary to the antisense strand, such that the two strands hybridize and form a duplex structure when combined under suitable conditions. In one embodiment, the iRNA can be or include a single strand of RNA that folds back on itself through self-complementarity to form a base-paired duplex that targets a transcript of interest. These are referred to as short hairpin RNAs or shRNAs, and can, if so desired, be encoded by a construct introduced to a cell. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive. Similarly, the region of complementarity to the target sequence is between 15 and 30 base pairs in length inclusive, more generally between 18 and 25 base pairs in length inclusive, yet more generally between 19 and 24 base pairs in length inclusive, and most generally between 19 and 21 base pairs in length nucleotides in length, inclusive. In some embodiments of any of the aspects, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length, as noted above.

Exemplary embodiments of types of inhibitory nucleic acids can include, e.g., siRNA, shRNA, miRNA, and/or amiRNA, which are known in the art. One of ordinary skill in the art can design and test an RNAi agent that targets PRPF31 mRNA. Publicly available RNAi design software permits one of skill in the art to select one or more sequences within a given target transcript that is or are likely to mediate efficient knock-down of target gene expression, and there are commercial sources for both design and preparation of RNAi agents. In some embodiments of any of the aspects, the RNAi molecule comprises the nucleic acid sequence of SEQ ID NO: 1 or SEQ ID NO: 2.

In some embodiments of any of the aspects, the RNA of an iRNA, e.g., a dsRNA, is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments of any of the aspects, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2- [known as a methylene (methylimino) or MMI backbone], —CH2-O—N(CH3)-CH2-, —CH2-N(CH3)-N(CH3)-CH2- and —N(CH3)-CH2-CH2-[wherein the native phosphodiester backbone is represented as —O—P—O—CH2-].

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

Modified RNAs can also contain one or more substituted sugar moieties. The iRNAs, e.g., dsRNAs, described herein can include one of the following at the 2′ position: OH; F; O-, S-, or N-alkyl; O-, S-, or N-alkenyl; O-, S- or N-alkynyl; or O-alkyl-Co-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO]mCH3, O(CH2).nOCH3, O(CH2)nNH2, O(CH2) nCH3, O(CH2)nONH2, and O(CH2)nON[(CH2)nCH3)]2, where n and m are from 1 to about 10. In some embodiments of any of the aspects, dsRNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments of any of the aspects, the modification includes a 2′ methoxyethoxy (2′-O—CH2CH2OCH3, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O-CH2-O-CH2-N(CH2)₂, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar.

An inhibitory nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Preparation of the modified nucleic acids, backbones, and nucleobases described above are known in the art.

Another modification of an inhibitory nucleic acid featured in the invention involves chemically linking the inhibitory nucleic acid to one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In one embodiment of any of the aspects, the agent that decreases PRPF31 is an antisense oligonucleotide, e.g., a nucleic acid with a sequence complementary to a target mRNA sequence. Antisense oligonucleotides are typically designed to block expression of a DNA or RNA target by hybridizing to the target and halting expression at the level of transcription, translation, or splicing. Antisense oligonucleotides as described herein are designed to hybridize to a target under typical intracellular conditions. Thus, oligonucleotides are chosen that are sufficiently complementary to the target, i.e., that hybridize sufficiently well and with sufficient specificity in the context of the cellular environment, to give the desired effect. For example, an antisense oligonucleotide that decreases the level of PRPF31 may comprise at least 10, at least 15, at least 20, at least 25, at least 30, or more bases complementary to a portion of the coding sequence of the human PRPF31 gene (e.g., SEQ ID NOs: 4-5), respectively.

In some embodiments of any of the aspects, the agent is an aptamer. Aptamers generally consist of relatively short oligonucleotides that typically range from 20 to 80 nucleotides in length, for example, at least 20 nucleotides, at least 30 nucleotides, at least 40 nucleotides, at least 50 nucleotides, at least 60 nucleotides, at least 70 nucleotides, or 80 nucleotides or more. An aptamer can be attached to a longer sequence, e.g., at one end or the other of the aptamer, although appended sequences that affect the secondary structure of the aptamer can affect aptamer function. The functional activity of an aptamer, i.e., binding to a given target molecule, involves interactions between moieties or elements in the aptamer with moieties or elements on the target molecule. Aptamers generally bind to specific targets through non-covalent interactions with a target, such as a protein, including but not limited to electrostatic interactions, hydrophobic interactions, and/or their complementary shapes. One of skill in the art can initially design an aptamer that targets PRPF31 using an in silco model known in the art, e.g., UNPACK, APTANI, 3D-DART, ModeRNA, or Unified Nucleic Acid Folding and hybridization package (UNAFold), or any other oligonucleotide structure prediction model. Following such design, the molecules can be synthesized and tested for binding and inhibitory activity as known in the art. Where desired, an aptamer can be expressed in a cell from a construct encoding the aptamer sequence.

The nucleic acids described herein that reduce the level or activity of PRPF31 can be commercially available, chemically synthesized using e.g., a nucleoside phosphoramidite or other approach, or isolated from a biological sample by DNA or RNA extraction methods. These isolation methods include but are not limited to column purification, ethanol precipitation, phenol-chloroform extraction, or acid guanidinium thiocyanate-phenol chloroform extraction (AGPC).

In certain embodiments, a vector is useful to express an agent described herein that reduces the levels or activity of PRPF31 in the in vitro-differentiated cells described herein, including but not limited to one or more polypeptides, peptides, ribozymes, peptide nucleic acids, siRNAs, or RNAi molecules, including for example, antisense oligonucleotides, antisense polynucleotides, siRNAs, shRNAs, micro-RNAs, and their antisense counterparts (e.g., antagoMiR)), antibodies, antigen binding fragments, or any combination thereof.

A vector is a nucleic acid construct designed for delivery to a host cell or for transfer of genetic material between different host cells. As used herein, a vector can be viral or non-viral. The term “vector” encompasses any genetic element that is capable of replication when associated with the proper control elements and that can transfer genetic material to cells. A vector can include, but is not limited to, a cloning vector, an expression vector, a plasmid, phage, transposon, cosmid, artificial chromosome, virus, virion, etc.

In some embodiments of any of the aspects, the vector is selected from the group consisting of: a plasmid and a viral vector.

An expression vector is a vector that directs expression of an RNA or polypeptide (e.g. an anti-PRPF31 antibody) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. “Expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene.

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

Non-integrative vectors include non-integrative viral vectors. Non-integrative viral vectors eliminate one of the primary risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. Containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free host cells. Other non-integrative viral vectors include adenoviral vectors and the adeno-associated viral (AAV) vectors.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages). This permits a self-limiting transient expression of a chosen heterologous gene or genes in a target cell.

Another example of a non-integrative vector is a minicircle vector. Minicircle vectors are circularized vectors in which the plasmid backbone has been released leaving only the eukaryotic promoter and cDNA(s) that are to be expressed.

As noted above, in some embodiments, the agent described herein is expressed in the cells from a viral vector. A “viral vector” includes a nucleic acid vector construct that includes at least one element of viral origin and has the capacity to be packaged into a viral vector particle. The viral vector can contain a nucleic acid encoding a polypeptide agent as described herein in place of non-essential viral genes. The vector and/or particle may be utilized for the purpose of transferring nucleic acids into cells either in vitro or in vivo.

In some embodiments, the nucleic acids and vectors described herein can be used to provide an antisense nucleic acid, a RNAi, an aptamer, or a vector comprising nucleic acids, to a cell in vitro or in vivo. The nucleic acids described herein can be delivered using any transfection reagent or other physical means that facilitates entry of nucleic acids into a cell. Methods and compositions for administering, delivering, or contacting a cell with a nucleic acid are known in the art, e.g., liposomes, nanoparticles, exosomes, nanocapsules, conjugates, alcohols, polylysine-rich compounds, arginine-rich compounds, calcium phosphate, microvesicles, microinjection and electroporation. An “agent that increases cellular uptake” is a molecule that facilitates transport of a molecule, e.g., nucleic acid, or peptide or polypeptide, or other molecule that does not otherwise efficiently transit the cell membrane across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), or a polyamine (e.g., spermine). Further examples of agents that increase cellular uptake are disclosed, for example, in Winkler (2013). Oligonucleotide conjugates for therapeutic applications. Ther. Deliv. 4(7); 791-809.

Assays known in the art can be used to test the efficiency of nucleic acid delivery to an in vitro-differentiated cell or tissue. Efficiency of introduction can be assessed by one skilled in the art by measuring mRNA and/or protein levels of a desired transgene (e.g., via reverse transcription PCR, western blot analysis, and enzyme-linked immunosorbent assay (ELISA)). In some embodiments, a vector described herein comprises a reporter protein that can be used to assess the expression of the desired transgene, for example by examining the expression of the reporter protein by fluorescence microscopy or a luminescence plate reader.

In some embodiments, the agent that reduces the levels or activity of PRPF31 is a nucleic acid encoding a polypeptide or a vector encoding a polypeptide. A polypeptide can encompass a singular “polypeptide” as well as plural “polypeptides,” and includes any chain or chains of two or more amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues provided herein are preferred to be in the “L” isomeric form. However, residues in the “D” isomeric form may be substituted for any L-amino acid residue provided the desired properties of the polypeptide are retained.

In some embodiments, the agent that reduces the level or activity of PRPF31 is a fusion polypeptide. In some embodiments, the agent that reduces the level or activity of PRPF31 is an antibody, an intrabody, a nucleic acid encoding an antibody, a nucleic acid encoding an intrabody, or a fragment thereof. In some embodiments, the antibody, intrabody, or fragment thereof, inhibits or reduces the assembly of the spliceosome by targeting PRPF31 in a cell.

An “antibody” as described herein encompasses any antibody or antibody fragment (i.e., a functional antibody fragment), or antigen-binding fragment that retains antigen-binding activity to a desired antigen or epitope, e.g., PRFP31. In one embodiment, the antibody or antigen-binding fragment thereof comprises an immunoglobulin chain or fragment thereof and at least one immunoglobulin variable domain sequence. Examples of antibodies include, but are not limited to, an scFv, a Fab fragment, a Fab′, a F(ab′)₂, a single domain antibody (dAb), a heavy chain, a light chain, a heavy and light chain, a full antibody (e.g., includes each of the Fc, Fab, heavy chains, light chains, variable regions etc.), a bispecific antibody, a diabody, a linear antibody, a single chain antibody, an intrabody, a monoclonal antibody, a chimeric antibody, or multimeric antibody. In addition, an antibody can be derived from any mammal, for example, primates, humans, rats, mice, llamas, horses, goats etc. In one embodiment, the antibody is human or humanized. In some embodiments, the antibody is a modified antibody. In some embodiments, the components of an antibody can be expressed separately such that the antibody self-assembles following expression of two or more protein components. In one embodiment, the antibody or antigen-binding fragment thereof comprises a framework region or an F_{c} region. An antibody fragment can retain 10-99% of the activity of the complete antibody (e.g., 10-90%, 10-80%, 10-70%, 10-60%, 10-50%, 10-40%, 10-30%, 10-20%, 50-99%, 50-90%, 50-80%, 50-70%, 50-60%, 20-99%, 30-99%, 40-99%, 60-99%, 70-99%, 80-99% 90-99% or any activity therebetween). It is also contemplated herein that a functional antibody fragment comprises an activity that is greater than the activity of the intact antibody (e.g., at least 2-fold or higher). In another embodiment, the antibody fragment comprises an affinity for its target that is substantially similar to the affinity of the intact antibody for the same target (e.g., epitope).

The antibody or immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art. Furthermore, in humans, the light chain can be a kappa chain or a lambda chain.

The antigen-binding domain of an antibody molecule is part of an antibody molecule, e.g., an immunoglobulin (Ig) molecule, that participates in antigen binding. The antigen binding site of an antibody is typically formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In a typical antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4. The CDRs within antibody variable regions confer antigen specificity and binding affinity. In general, there are three CDRs in each heavy chain variable region (HCDR1, HCDR2, HCDR3) and three CDRs in each light chain variable region (LCDR1, LCDR2, LCDR3). The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme). The CDRs defined according the “Chothia” number scheme are also sometimes referred to as “hypervariable loops.” For example, under Kabat, the CDR amino acid residues in the human heavy chain variable domain (VH) are numbered 31-35 (HCDR1), 50-65 (HCDR2), and 95-102 (HCDR3); and the CDR amino acid residues in the human light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). Each VH and VL typically includes three CDRs and four FRs, arranged from amino-terminus to carboxy-terminus in the following order: FR1, CDR1, FR2, CDR2, FR3, CDR3, FR4.

A full length antibody is generally an immunoglobulin (Ig) molecule (e.g., an IgG, IgE, IgM antibody), for example, that is naturally occurring, and formed by normal immunoglobulin gene fragment recombinatorial processes.

A functional antibody fragment or antigen-binding fragment binds to the same antigen or epitope as that recognized by an intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. In some embodiments, the functional antibody fragment retains at least 20% of the activity of the intact or full-length antibody, for example, as assessed by measuring the degree of inhibition of the target protein (e.g., PRPF31). In other embodiments, the functional antibody fragment retains at least 30%, at least 40%, at least 50%, at least 60%, at least 70%, at least 80%, at least 90%, at least 95%, at least 98%, at least 99%, or even 100% (i.e., substantially similar) activity to the intact antibody. It is also contemplated herein that a functional antibody fragment will comprise increased activity as compared to the intact antibody (e.g., at least 1-fold, at least 2-fold, at least 5-fold, at least 10-fold, at least 100-fold or more).

When an intrabody is desired, i.e., an antibody expressed in a cell to target an intracellular antigen, e.g., PRPF31, the nucleic acid or gene encoding the anti-PRPF31 antibody or fusion protein typically does not encode a secretory sequence. An intrabody can include an scFv. In some instances, it can encode a secretory sequence but also has an intended targeting sequence. In other embodiments, the intrabody genes encode another intracellular targeting sequence, e.g., a nuclear localization sequence. Thus the intrabodies can be directed to a particular cellular compartment by incorporating signaling motifs, such as a C-terminal ER retention signal, a mitochondrial targeting sequence, a nuclear localization sequence, etc.

In some embodiments, the agent that reduces the levels or activity of PRPF31 is a dominant negative mutant of PRPF31 or a PRPF31 comprising one or more point mutations. PRPF31 mutations of this kind are known in the art and described, e.g., by Vithana et al., Mol Cell. (2001); Deery et al. Hum Mol Gen. (2002); Waseem et al. Invest. Ophtal. Vis. Sci. (2007); and Rio Frio Clin Invest. (2008), each of which are incorporated herein by reference in their entireties.

Transplant Compositions

In one aspect, described herein is a method of promoting survival and/or engraftment of transplanted human, in vitro-differentiated cells, the method comprises contacting, human in vitro-differentiated cells with an agent that decreases the level or activity of PRPF31, and transplanting the cells into a tissue of a subject in need thereof. In some embodiments, the in-vitro differentiated cells are of a mesodermal lineage. In some embodiments, the in vitro-differentiated cells are cardiomyocytes. The in vitro-differentiated cells can be any of those described above, or other mesodermal lineage cells differentiated in vitro as known herein in the art.

For the treatment of cells with an agent that decreases the level or activity of PRPF31, the formulation, dosage and timing of the treatment with the agent will vary with the nature of the agent. For example, a small molecule or other agent that crosses the cell's plasma membrane can simply be administered to the culture medium in which the cells are maintained, while a small molecule or other agent that does not readily cross the plasma membrane can be formulated with a moiety that facilitates delivery into the cell. The factors that determine whether a given agent will transit the plasma membrane on its own, e.g., by passive transport, or whether it will require formulation with another agent or entity that promotes or facilitates membrane transit are discussed, for example, in a review article “Getting Across the Cell Membrane: An Overview for Small Molecules, Peptides, and Proteins,” by Yang & Hinner, Methods Mol. Biol. 1266: 29-53 (2015), which is incorporated herein by reference in its entirety. The authors note that small, nonpolar gases such as oxygen, carbon dioxide and nitrogen and small polar molecules such as ethanol readily cross membranes, but that even slightly larger metabolites such as urea and glycerol have lower permeability, and the plasma membrane is virtually impermeable to larger, uncharged polar molecules and all charged molecules, including ions. Thus, approaches that engage other mechanisms need to be considered for many peptides, polypeptides, oligo- or polynucleotides and many organic compounds and small molecules.

Many molecules, including sugars (glucose, galactose, fructose), amino acids and nucleotides are transported across the cell membrane by membrane transporter proteins. Conjugating an agent one wishes to transport across the membrane with a natural substrate for a transporter protein can be effective for delivery of some agents to the cytosol. See, e.g., Dahan et al., Expert Opin. Drug Deliv. 9: 1001-1013 (2012), and Majumdar et al., Adv. Drug Deliv. Rev. 56: 1437-1452 (2004), each of which is incorporated herein by reference.

Limited mechanical disruption of the membrane can be useful to introduce agents ranging from small molecules to proteins into cells. Thus, electroporation, devices that force cells through microfluidic channels in a solution containing the desired agent (see, e.g., Sharei et al., Proc. Natl. Acad. Sci. U.S.A. 110: 2082-2087 (2013)), and silicon nanowires that pierce the cell membrane (Shalek et al., Proc. Natl. Acad. Sci. U.S.A. 107: 1870-1875 (2010)) can promote uptake of an agent by cultured cells.

Conjugation of an agent to a cell-penetrating peptide (CPP) can also promote uptake of macromolecules, including proteins. Examples of CPPS include the viral TAT peptide (see, e.g., Fawell et al., Proc. Natl. Acad. Sci. U.S.A. 91: 664-668 (1994), Nagahara et al., Nat. Med. 4: 1449-1452 (1998), and Langel, Handbook of cell-penetrating peptides. 2^{11d}. Boca Raton: CRC Press (2010)), and the amphiphilic Pep-1 peptide (see, e.g., Morris et al., Nat. Biotechnol. 19: 1173-1176 (2001)). Other proteins that can promote uptake of a conjugated cargo protein agent include, for example, the autoantibody 3E10, which can translocate across the cell membrane, and has been proposed to penetrate into the nucleus (see, e.g., Hansen et al., Sci. Transl. Med. 4 157ra142 (2012)) and shown to deliver an exogenous phosphatase enzyme across the cell membrane (see, e.g., Lawlor et al., Hum. Mol. Genet. 22: 1525-1538 (2013)). Alternatively, packaging protein agents in virus-like particles or attaching them to an engineered bacteriophage T4 head has been reported to promote cytosolic delivery (see, e.g., Kaczmarczyk et al., Proc. Natl. Acad. Sci. U.S.A. 108: 16998-17003 (2011), and Tao et al., Proc. Natl. Acad. Sci. U.S.A. 110: 5846-5851 (2013)). Each of the references cited is incorporated herein by reference.

Lipid and polymer-based formulations for delivery of an agent across the cell membrane include those that encapsulate the agent in liposomes or that complex the agent with lipids. Such approaches are well established for introducing nucleic acids (e.g., siRNAs, antisense oligonucleotides, ribozymes, aptamers, constructs encoding protein agents, shRNAs, antisense expression cassettes, aptamers etc.) to cells. Commercial preparations for lipofection are readily available, e.g., LIPOFECTAMINE™ (ThermoFisher Scientific) transfection reagents, among others. A mixture of cationic and neutral lipids has been reported to translocate negatively charged proteins (see, e.g., Zelphati et al., J. Biol. Chem. 276: 35103-35110 (2001) and Torchilin, Drug Discov. Today Technol. 5: e95-e103 (2008), each of which is incorporated herein by reference). Polymer-based formulations including polyethylenimine (PEI) and poly-β-amino ester nanoparticles enhance endosomal escape of cargos including proteins when administered to cells (see, e.g., Behr, Chim. Int. J. Chem. 51: 34-36 (1997), and Su et al., Biomacromolecules 14: 1093-1102 (2013), each of which is incorporated herein by reference). Further examples of delivery formulations include but are not limited to multilamellar vesicles (MLV), unilamellar vesicles (UMVs), PEG-coated liposomes, exosomes, nanoparticles, and FuGENE® (Promega Corporation, Madison Wis.).

Any of these or other approaches or formulations known in the art can be applied to a given agent for introduction of an agent that decreases the level or activity of PRPF31 to in vitro-differentiated cells as described herein.

In the context of delivering an agent described herein, the term “contacting,” “delivering” or “delivery” is intended to encompass both delivery of an agent that reduces the levels or activity of PRPF31 from outside the cell, and delivery from within the cell, e.g., by expression from a nucleic acid construct or vector. For example, agents described herein can be introduced from outside the cell by adding the agent to the cell culture medium in which in vitro-differentiated cells as described herein are maintained or grown. Alternatively, the agents described herein can be delivered by expression within the cell from an exogenous construct, e.g., a viral or other expression vector. Such a construct can be episomal or stably integrated within the cell's genome. In one embodiment, the step of contacting an in vitro-differentiated mesodermal lineage cell or cardiomyocyte with an agent described herein comprises the use of cells that stably express the agent from a construct. In another embodiment, the step of contacting an in vitro-differentiated cell or cardiomyocyte with an agent described herein comprises the use of cells that transiently express the agent from a construct.

With respect to dosage, the amount to use of an agent that decreases the level or activity of PRPF31 will depend upon the nature of the agent and the formulation. Thus, agents that transit cell membranes without requiring conjugation or complex formation with another agent can be applied to cultured cells at picomolar to micromolar concentrations which can be optimized in a straightforward manner via a dose response titration. Agents that require conjugation or complex formation with another agent for transmembrane delivery can also be titrated over a range of concentrations for effective knockdown of PRPF31 mRNA, protein or activity. Once a working range that knocks down the level or activity of the PRPF31 is identified, in vivo experiments in which treated cells are injected or otherwise administered to, for example, an animal model can be used to identify the dosage that provides the best results for survival and/or engraftment.

siRNA that targets PRPF31 (e.g., SEQ ID NO: 1) at a concentration of 5 nanomolar (nM) is demonstrated in the Examples herein to provide beneficial effects on in vitro-differentiated cardiomyocytes when introduced via lipofection. In practice, the concentration can vary, e.g., between 0.5 nM to 50 nM, or any concentration therebetween.

With respect to timing, the duration of treatment of cells with a given agent or formulation and the timing of such treatment relative to the administration of the treated cells to the subject can also vary with the nature of the agent and the nature of the cells (e.g., cardiomyocytes vs kidney, bone or other mesodermal lineage cell type). However, one of ordinary skill in the art can determine for a given agent and formulation how long to treat the cells to achieve optimal PRPF31 inhibition or knockdown, and how far in advance of cell administration to the subject to initiate the treatment of the cells. In general, agents that take longer to achieve knockdown or inhibition should be administered earlier with respect to the planned time of cell administration. In some embodiments of any of the aspects, the in vitro-differentiated cells are contacted with an agent that decreases the levels or activity of PRPF31 in the range of 1-48 hours prior to administration of the cells to a subject, e.g., 1-36 hours, 1-24 hours, 1-18 hours, 1-12 hours, 1-6 hours, 1-4 hours or 1-2 hours before the cells are to be administered to a subject. In some embodiments of any of the aspects, the cells are contacted with the agent that decreases the levels or activity of PRPF31 at least 1 hour before, at least 2 hours before, at least 3 hours before, at least 4 hours before, at least 6 hours before, at least 8 hours before, at least 10 hours before, at least 12 hours before, at least 14 hours before, at least 16 hours before, at least 18 hours before, at least 24 hours before, at least 30 hours before, at least 36 hours before, at least 42 hours before, or at least 48 hours before the cells are administered to a subject.

Transplant compositions as described herein comprise in vitro-differentiated cells treated with an agent that decreases the level or activity of PRPF31 in those cells, in admixture with a pharmaceutically acceptable carrier. The transplant composition can be formulated, for example, for administration by injection to a tissue or organ in need of repair or functional augmentation. Alternatively, the transplant composition can be formulated on or in a scaffold as described herein or as known in the art, e.g., to assist with retaining the transplanted cells in a given physical location or to further augment survival and/or engraftment. Cells associated with a scaffold can also be formulated for injection, e.g., where the scaffold is a gel or other matrix with a fluid consistency. Alternatively, where the scaffold is more solid, cells associated with a scaffold can be formulated to apply to a tissue or organ or to implant surgically into or onto a tissue or organ.

One of skill in the art can determine the number of cells needed for a transplant or graft depending, for example, upon the extent of damage to be repaired and the cell type. For example, in vitro-differentiated cardiomyocytes as described herein can be administered to a subject in need of improved cardiac function. In some embodiments, about 10 million to about 10 billion cardiomyocytes are administered to the subject. For use in the various aspects described herein, an effective amount of human cardiomyocytes can comprise at least 1×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 3×10⁸, at least 4×10⁸, at least 5×10⁸, at least 6×10⁸, at least 7×10⁸, at least 8×10⁸, at least 9×10⁸, at least 1×10⁹, at least 2×10⁹, at least 3×10⁹, at least 4×10⁹, at least 5×10⁹, at least 6×10⁹, at least 7×10⁹, at least 8×10⁹, at least 9×10⁹, at least 1×10⁹, at least 1×10¹⁰ or more cells for transplant or graft. Similar numbers of other in vitro-differentiated mesoderm lineage cells can be used for transplant or graft to different tissues.

While the cells described herein for graft or transplant are generally fully differentiated, they can have limited proliferative potential, meaning that long-term survival and/or engraftment is preferred, and the treatment to decrease the level or activity of PRPF31 in the cells can promote such survival and engraftment. It is also contemplated that cells differentiated in vitro from pluripotent stem cells to a stem or precursor cell of the mesodermal lineage upstream developmentally from a desired cell type can, in some embodiments, be treated as described herein to decrease the level or activity of PRPF31 and administered, such that the treated cells expand in number and differentiate after administration to the subject.

The transplant compositions described herein will, in some embodiments, lack or substantially lack the agent that decreases the level of PRPF31. That is, the cells can be treated transiently in vitro with the agent, then formulated for transplant without the agent. By “substantially lack” in this context, the transplant composition or formulation would have only that agent that remains in the cells after treatment and before or during administration. It is not necessarily required, but in some embodiments, and depending upon the nature of the agent and the delivery formulation used with it, it can be advantageous to wash out or remove the agent from adherent cells in culture prior to formulation for transplant. In other embodiments, it is contemplated that the cells can be formulated and administered in a transplant composition that includes the agent, for example in solution or suspension with the cells.

Scaffold Compositions:

In one aspect, the in vitro-differentiated cells described herein can be admixed with or grown in or on a preparation that provides a scaffold or substrate to support the cells. A scaffold is a structure comprising a biocompatible material including but not limited to a gel, sheet, matrix or lattice that can contain cells in a desired location but permit the entry or diffusion of factors in the environment necessary for survival and function. A number of biocompatible polymers suitable for a scaffold are known in the art.

Such a scaffold or substrate can provide a physical advantage in securing the cells in a given location, e.g., after implantation, as well as a biochemical advantage in providing, for example, extracellular cues for the further maturation or, e.g., maintenance of phenotype until the cells are established.

Biocompatible synthetic, natural, as well as semi-synthetic polymers can be used for synthesizing polymeric particles that can be used as a scaffold material. In general, for the practice of the methods described herein, it is preferable that a scaffold biodegrades such that the in vitro-differentiated cells can be isolated from the polymer prior to implantation or such that the scaffold degrades over time in a subject and does not require removal. Thus, in one embodiment, the scaffold provides a temporary structure for growth and/or delivery of in vitro-differentiated cells to a subject in need thereof. In some embodiments, the scaffold permits human cells to be grown in a shape suitable for transplantation or administration into a subject in need thereof, thereby permitting removal of the scaffold prior to implantation and reducing the risk of rejection or allergic response initiated by the scaffold itself.

Examples of polymers which can be used include natural and synthetic polymers, although synthetic polymers are preferred for reproducibility and controlled release kinetics. Synthetic polymers that can be used include biodegradable polymers such as poly(lactide) (PLA), poly(glycolic acid) (PGA), poly(lactide-co-glycolide) (PLGA or PLA/PGA copolymer), and other polyhydroxyacids, poly(caprolactone), polycarbonates, polyamides, polyanhydrides, polyphosphazene, polyamino acids, polyortho esters, polyacetals, polycyanoacrylates and biodegradable polyurethanes; non-biodegradable polymers such as polyacrylates, ethylene-vinyl acetate polymers and other acyl-substituted cellulose acetates and derivatives thereof; polyurethanes, polystyrenes, polyvinyl chloride, polyvinyl fluoride, poly(vinyl imidazole), chlorosulphonated polyolefins, and polyethylene oxide. Examples of biodegradable natural polymers include proteins such as albumin, collagen, fibrin and silk, polysaccharides such as alginate, heparin and other naturally occurring biodegradable polymers of sugar units. Alternatively, combinations of the aforementioned polymers can be used. In one aspect, a natural polymer that is not generally found in the extracellular matrix can be used.

PLA, PGA and PLA/PGA copolymers are particularly useful for forming biodegradable scaffolds. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. Methods of preparing polylactides are well documented in the patent literature. The following U.S. Patents, the teachings of which are hereby incorporated by reference, describe in detail suitable polylactides, their properties and their preparation: U.S. Pat. No. 1,995,970 to Dorough; U.S. Pat. No. 2,703,316 to Schneider; U.S. Pat. No. 2,758,987 to Salzberg; U.S. Pat. No. 2,951,828 to Zeile; U.S. Pat. No. 2,676,945 to Higgins; and U.S. Pat. Nos. 2,683,136; 3,531,561 to Trehu.

PGA is a homopolymer of glycolic acid (hydroxyacetic acid). In the conversion of glycolic acid to poly(glycolic acid), glycolic acid is initially reacted with itself to form the cyclic ester glycolide, which in the presence of heat and a catalyst is converted to a high molecular weight linear-chain polymer. PGA polymers and their properties are described in more detail in Cyanamid Research Develops World's First Synthetic Absorbable Suture”, Chemistry and Industry, 905 (1970).

Fibers can be formed by melt-spinning, extrusion, casting, or other techniques well known in the polymer processing area. Preferred solvents, if used to remove a scaffold prior to implantation, are those which are completely removed by the processing or which are biocompatible in the amounts remaining after processing.

Polymers for use in the matrix should meet the mechanical and biochemical parameters necessary to provide adequate support for the cells with subsequent growth and proliferation. The polymers can be characterized with respect to mechanical properties such as tensile strength using an Instron tester, for polymer molecular weight by gel permeation chromatography (GPC), glass transition temperature by differential scanning calorimetry (DSC) and bond structure by infrared (IR) spectroscopy.

The substrate or scaffold can be nanopatterned or micropatterned with grooves and ridges that permit growth and promote maturation of cardiac cells or tissues on the scaffold. Scaffolds can be of any desired shape and can comprise a wide range of geometries that are useful for the methods described herein. A non-limiting list of shapes includes, for example, patches, hollow particles, tubes, sheets, cylinders, spheres, and fibers, among others. The shape or size of the scaffold should not substantially impede cell growth, cell differentiation, cell proliferation or any other cellular process, nor should the scaffold induce cell death via e.g., apoptosis or necrosis. In addition, care should be taken to ensure that the scaffold shape permits appropriate surface area for delivery of nutrients from the surrounding medium to cells in the population, such that cell viability is not impaired. The scaffold porosity can also be varied as desired by one of skill in the art.

In some embodiments, attachment of the cells to a polymer is enhanced by coating the polymers with compounds such as basement membrane components, fibronectin, agar, agarose, gelatin, gum arabic, collagen type I, II, III, IV, and V, laminin, glycosaminoglycans, polyvinyl alcohol, mixtures thereof, and other hydrophilic and peptide attachment materials known to those skilled in the art of cell culture or tissue engineering. Examples of a material for coating a polymeric scaffold include polyvinyl alcohol and collagen. As will be appreciated by one of skill in the art, Matrigel™ is not suitable for administration to a human subject, thus the compositions described herein do not include Matrigel™.

In some embodiments it can be desirable to add bioactive molecules/factors to the scaffold. A variety of bioactive molecules can be delivered using the matrices described herein.

In one embodiment, the bioactive factors include growth factors. Examples of growth factors include platelet derived growth factor (PDGF), transforming growth factor alpha or beta (TGFβ), bone morphogenic protein 4 (BMP4), fibroblastic growth factor 7 (FGF7), fibroblast growth factor 10 (FGF10), epidermal growth factor (EGF/TGFβ), vascular endothelium growth factor (VEGF), some of which are also angiogenic factors. These factors are known to those skilled in the art and are available commercially or described in the literature. Bioactive molecules can be incorporated into the matrix and released over time by diffusion and/or degradation of the matrix, or they can be suspended with the cell suspension.

Pharmaceutically Acceptable Carriers:

The in vitro-differentiated cells treated with an agent that decreases the level or activity of PRPF31 can be formulated for transplant by admixture with a pharmaceutically acceptable carrier. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as toxicity, transplant rejection, allergic reaction, and the like. A pharmaceutically acceptable carrier will not promote the raising of an immune response to an agent with which it is admixed, unless so desired.

In general, the compositions comprising in vitro-differentiated cells described herein are administered as liquid suspension formulations including the cells in combination with the pharmaceutically acceptable carrier. One of skill in the art will recognize that a pharmaceutically acceptable carrier to be used in a transplant composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the cells as described herein using routine experimentation.

Transplant compositions can optionally contain additional bioactive ingredients that further promote the survival, engraftment or function of the administered cells or, optionally, the tissue, organ or subject to which the composition is administered. Examples include, but are not limited to growth factors, nutrients, analgesics, anti-inflammatories and small molecule drugs, such as kinase activators, among others.

Physiologically tolerable carriers for the suspension of cells for a transplant composition include sterile aqueous physiological saline solutions that contain no additional materials other than the cells, or that contain a buffer such as sodium phosphate at physiological pH value, such as phosphate-buffered saline. Still further, aqueous carriers can contain more than one buffer salt, as well as salts such as sodium and potassium chlorides, dextrose, polyethylene glycol and other solutes.

Administration and Efficacy

Described herein are compositions and methods that promote the survival and/or engraftment of transplanted, in vitro-differentiated human cells, including cells of the mesodermal lineage, including, but not limited to cardiomyocytes. Transplantation of cells treated with an agent that decreases the level or activity of PRPF31 can involve the injection of a transplant composition comprising cells in a suspension, with or without a matrix or scaffold, into a desired location, e.g., a tissue in need of repair. Alternatively, transplantation can involve the surgical placement of a transplant composition comprising cells in a matrix or on a scaffold, onto or into a desired location, tissue or organ, e.g., a tissue or organ in need of repair.

The survival or engraftment of transplanted cells can be determined by any method known in the art, for example, by monitoring tissue or organ function following transplantation. Measured or measurable parameters for efficacy include clinically detectable markers of function or disease, for example, elevated or depressed levels of a clinical or biological marker, functional parameters, as well as parameters related to a clinically accepted scale of symptoms or markers for health or a disease or disorder. The survival and engraftment of the transplanted cells can be quantitatively or qualitatively determined by histological and molecular methods. In one approach, survival and engraftment can be evaluated in an appropriate animal model, e.g., a NOD scid gamma mouse model as discussed in the Examples herein. Using such a model, human cells can be injected and then evaluated for survival and engraftment by measuring human specific markers in the recipient tissue, e.g., cardiac tissue. In brief, measurement of the number of cells injected versus the number engrafted provides a measure of engraftment efficiency. Measurement of viable transplanted cells in the tissue provides a measure of survival. Viability of engrafted cells can be determined or measured by any of several methods, including, for example, histology and/or immunohistochemistry for human markers. The identification of cells as being from the transplant is based on the presence of human markers, and the morphology of the cells and/or their organization in the tissue can indicate cell viability. As but one example, Masson elastic trichrome or Movat pentachrome histological stains are particularly useful to assess interstitial fibrosis, cardiomyocyte necrosis and disarray, in addition to the presence of contraction bands in cardiac tissues. Alternatively, one can use laser capture microdissection and quantitation of human DNA sequence (e.g., human ALU repeat sequence). As yet another alternative for the evaluation of graft survival, one can quantitate human DNA sequence in homogenized tissue, e.g., heart tissue. For example, cells, e.g., cardiomyocytes treated with or without an inhibitor of PRPF31 can be transplanted into tissue, e.g., cardiac tissue, of a plurality of mice. At selected timepoints after transplant, tissue from individual mice can be harvested and evaluated for the presence and/or amount of human DNA as measure of the maintenance or persistence of the transplanted cells.

The term “effective amount” as used herein refers to the amount of a population of in vitro-differentiated cells treated as described herein needed to alleviate at least one or more symptoms of a disease or disorder, including but not limited to an injury, disease, or disorder. An “effective amount” relates to a sufficient amount of a composition to provide the desired effect, depending upon the cell type administered and the disease or disorder addressed, e.g., the amount necessary to treat a subject having an infarct zone following myocardial infarction, improve cardiomyocyte engraftment, prevent onset of heart failure following cardiac injury, enhance vascularization of a graft, enhance renal function, etc. The term “therapeutically effective amount” therefore refers to an amount of human in vitro-differentiated cells treated with an agent that decreases PRPF31 level or activity, or a composition including such cells that is sufficient to promote a particular effect when administered to a typical subject, such as one who has, or is at risk for, a cardiac disease, among others. An effective amount as used herein also includes an amount sufficient to prevent or delay the development of a symptom of the disease, alter the course of a disease symptom (for example but not limited to, slow the progression of a symptom of the disease), or reverse a symptom of the disease. It is understood that for any given case, an appropriate “effective amount” can be determined by one of ordinary skill in the art using routine experimentation.

In some embodiments, the subject is first diagnosed as having a disease or disorder affecting a tissue or organ comprising cells of the type differentiated in vitro, prior to administering the cells according to the methods described herein. In some embodiments, the subject is first diagnosed as being at risk of developing a disease (e.g., heart failure following myocardial injury or kidney disease) or disorder prior to administering the cells.

As noted above, for use in the various aspects described herein, an effective amount of human cardiomyocytes is at least 1×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 3×10⁸, at least 4×10⁸, at least 5×10⁸, at least 6×10⁸, at least 7×10⁸, at least 8×10⁸, at least 9×10⁸, at least 1×10⁹, at least 2×10⁹, at least 3×10⁹, at least 4×10⁹, at least 5×10⁹, at least 6×10⁹, at least 7×10⁹, at least 8×10⁹, at least 9×10⁹, at least 1×10⁹, at least 1×10¹⁰ or more cells for transplant or graft. Similar numbers of other in vitro-differentiated mesoderm lineage cells can be used for transplant or graft to different tissues. Effective amounts of cells or a transplant composition comprising them can be initially estimated through use of an appropriate animal model. As but one example, murine, canine and porcine models of cardiac infarction are known and can be used to gauge the amounts of cells or transplant compositions comprising them effective for treatment.

In some embodiments, a composition comprising human in vitro-differentiated cells treated with an agent that decreases PRPF31 level or activity permits engraftment of the cells in the desired tissue or organ at an efficiency at least 20% greater than the engraftment when such cells are administered without such treatment; in other embodiments, such efficiency is at least 30%, at least 40%, at least 50%, at least 60%, at least 70%, at least 80%, at least 90%, at least 1-fold, at least 2-fold, at least 5-fold, at least 10-fold, at least 100-fold or more than the efficiency of engraftment when cells are administered without such treatment.

When the cells are in vitro-differentiated cardiomyocytes, an effective amount of cardiomyocytes is administered to a subject by intracardiac administration or delivery. In this context, “intracardiac” administration or delivery refers to all routes of administration whereby a population of cardiomyocytes is administered in a way that results in direct contact of these cells with the myocardium of a subject, including, but not limited to, direct cardiac injection, intra-myocardial injection(s), intra-infarct zone injection, ischemic- or peri-ischemic zone injection, injection into areas of wall thinning, injection into areas at risk for maladaptive cardiac remodeling, injection or implantation during surgery (e.g., cardiac bypass surgery, during implantation of a cardiac mini-pump or a pacemaker, etc.). In some such embodiments, the cells are injected into the myocardium (e.g., cardiomyocytes), or into the cavity of the atria and/or ventricles. In some embodiments, intracardiac delivery of cells includes administration methods whereby cells are administered, for example as a cell suspension, to a subject undergoing surgery via a single injection or multiple “mini” injections into the desired region of the heart.

The choice of formulation will depend upon the specific composition used and the number of treated cells to be administered; such formulations can be adjusted by the skilled practitioner. However, as an example, where the composition includes cardiomyocytes in a pharmaceutically acceptable carrier, the composition can be a suspension of the cells in an appropriate buffer (e.g., saline buffer) at an effective concentration of cells per mL of solution. The formulation can also include cell nutrients, a simple sugar (e.g., for osmotic pressure regulation) or other components to maintain the viability of the cells. Alternatively, as noted herein above, the formulation can comprise a scaffold, such as a biodegradable scaffold as described herein or as known in the art.

In some embodiments, additional agents to aid in treatment of the subject can be administered before or following treatment with the cells as described. Such additional agents can be used, for example, to prepare the target tissue for administration of the cells. Alternatively, the additional agents can be administered after the cells to support the engraftment and growth or integration of the administered cells into the tissue or organ. In some embodiments, the additional agent comprises growth factors, such as VEGF, PDGF, FGF, aFGF, bFGF, IGF or Notch signaling compounds. Other exemplary agents can be used, for example, to reduce the load on the heart while cardiomyocytes are engrafting (e.g., beta blockers, medications to lower blood pressure, etc.).

In some embodiments of any of the aspects, the additional agent is administered beginning at least 1 hour, at least 5 hours, at least 10 hours, at least 15 hours, at least 20 hours, at least 1 day, at least 2 days, at least 3 days, at least 4 days, at least 5 days, at least 6 days, at least 7 days at least 8 days, at least 9 days, at least 10 days, prior to administration of the treated cells. In some embodiments of any of the aspects, the additional agent is administered concurrently with or following administration of the treated cells.

The efficacy of treatment can be determined by the skilled clinician. However, a treatment is considered “effective treatment,” as the term is used herein, if any one or all of the symptoms, or other clinically accepted symptoms or markers of disease, e.g., cardiac disease, heart failure, cardiac injury or a cardiac disorder, renal disease or disorder, etc. are reduced, e.g., by at least 10% and including, for example, at least 20%, at least 30%, at least 40%, at least 50%, at least 60%, at least 70%, at least 80%, at least 90% or more following administration of a transplant composition comprising treated cells as described herein. Methods of measuring these indicators are known to those of skill in the art and/or described herein.

Where the transplanted cells are cardiomyocytes, indicators of a cardiac disease or cardiac disorder, or cardiac injury include functional indicators or parameters, e.g., stroke volume, heart rate, left ventricular ejection fraction, heart rhythm, blood pressure, heart volume, regurgitation, etc. as well as biochemical indicators, such as a decrease in markers of cardiac injury, such as serum lactate dehydrogenase, or serum troponin, among others. As one example, myocardial ischemia and reperfusion are associated with reduced cardiac function. Subjects that have suffered an ischemic cardiac event and/or that have received reperfusion therapy have reduced cardiac function when compared to that before ischemia and/or reperfusion. Measures of cardiac function include, for example, ejection fraction and fractional shortening. Ejection fraction is the fraction of blood pumped out of a ventricle with each heartbeat. The term ejection fraction applies to both the right and left ventricles. LVEF refers to the left ventricular ejection fraction (LVEF). Fractional shortening refers to the difference between end-diastolic and end-systolic dimensions divided by end-diastolic dimension.

Non-limiting examples of clinical tests that can be used to assess cardiac functional parameters include echocardiography (with or without Doppler flow imaging), electrocardiogram (EKG), exercise stress test, Holter monitoring, or measurement of natriuretic peptide (e.g., atrial natriutetic peptide).

Where necessary or desired, animal models of injury or disease can be used to gauge the effectiveness of a particular composition as described herein. For example, an isolated working rabbit or rat heart model, or a coronary ligation model in either canines or porcines can be used. Animal models of cardiac function are useful for monitoring infarct zones, coronary perfusion, electrical conduction, left ventricular end diastolic pressure, left ventricular ejection fraction, heart rate, blood pressure, degree of hypertrophy, diastolic relaxation function, cardiac output, heart rate variability, and ventricular wall thickness, etc.

For the monitoring of engraftment or survival of transplanted cells, the cells can be marked or tagged, for example, by introduction of a construct that directs the expression of a marker, such as, but not limited to GFP or other fluorescent protein, or an epitope tag. When cells expressing such a marker are administered to an animal model, functional parameters can be gauged as for any cell, but tissue can also be removed after cell administration and tested or assayed, e.g., via fluorescence microscopy or immunohistochemistry, for the expression of the marker. Persistence or level of marker expression can thus be used to gauge the efficacy of the cell treatment described herein in enhancing or promoting cell survival and/or engraftment using such an animal model.

In addition to treatment of cells with an agent that decreases the level or activity of PRPF31, when the cells are cardiomyocytes, other approaches or treatments known in the art to promote or enhance the survival, engraftment, maturity and/or function of transplanted cardiomyocytes can be performed before, concurrently or in parallel with, or after administration of the treated cells. See, for example, WO2018/170280, which describes, among other things, the in vitro differentiation and co-transplantation of epicardial cells with in vitro-differentiated cardiomyocytes. Such treatment was also found to promote cardiomyocyte engraftment and to enhance cardiac function upon transplant. WO2018/170280 is incorporated herein by reference in its entirety, but with particular note of methods described therein for transplant of cardiomyocytes, markers and measurement of cardiomyocyte maturity, co-transplant with epicardial cells, measurement of transplant engraftment, survival and/or function, and the measurement of efficacy of such transplants.

In other embodiments, the transplant compositions described herein may be used to treat a disease or improve survival, e.g., to reduce the onset, incidence of severity of a cardiovascular disease. The efficacty of a therapeutic treatment can be assessed by the presence or absence of a symptom of a disease by functional output (e.g., measuring cardiac output or renal function), markers, levels or expression (e.g., serum levels of cardiac enzymes, markers of ischemia, renal function or insufficiency), and/or electrographic means (e.g., an electrocardiogram). Further, as will be appreciated by a skilled physician, the ability to modify the transplant compositions described herein can permit them to customize a treatment based on a subject's particular set of symptoms and/or severity of disease and further to minimize side effects or toxicity.

Some embodiments of the compositions and methods described herein can be defined according to any of the following numbered paragraphs:
1. A composition comprising human cells differentiated in vitro from stem cells and an agent that decreases the level or activity of Pre-mRNA Processing Factor 31 (PRPF31).
2. The composition of paragraph 1, wherein the cells differentiated in vitro from stem cells are cardiomyocytes.
3. The composition of any one of paragraphs 1-2, wherein the cells differentiated in vitro from stem cells are of a mesodermal lineage.
4. The composition of any one of paragraphs 1-3, wherein the in vitro-differentiated cells are of a cell type selected from: cardiomyocytes, skeletal muscle cells, smooth muscle cells, kidney cells, endothelial cells, skin cells, adrenal cortex cells, bone cells, white blood cells, and microglial cells.
5. The composition of any one of paragraphs 1-4, wherein the in vitro-differentiated human cells are differentiated from induced pluripotent stem cells (iPSCs) or from embryonic stem cells.
6. The composition of any one of paragraphs 1-5, wherein the stem cells are derived from a healthy subject.
7. The composition of any one of paragraphs 1-6, wherein the agent is a small molecule, a polypeptide, a nucleic acid molecule or a vector comprising a nucleic acid molecule.
8. The composition of any one of paragraphs 1-7, wherein the agent comprises or encodes a nucleic acid molecule comprising an antisense sequence, an aptamer or an RNA interference molecule (RNAi) that targets PRPF31 or its RNA transcript.
9. The composition of paragraph 7, wherein the vector is selected from the group consisting of: a plasmid and a viral vector.
10. The composition of paragraph 8, wherein the RNAi molecule comprises the nucleic acid sequence of SEQ ID NO: 1.
11. A transplant composition for transplant to a recipient, the composition comprising in vitro-differentiated human cardiomyocytes that have been contacted with an agent that decreases the level or activity of PRPF31, and a pharmaceutically acceptable carrier.
12. The transplant composition of paragraph 11, wherein the agent is selected from a small molecule, a polypeptide, a nucleic acid molecule or a vector comprising a nucleic acid molecule.
13. The transplant composition of any one of paragraphs 11-12, wherein the agent comprises or encodes a nucleic acid molecule comprising an antisense sequence, an aptamer or an RNA interference molecule (RNAi) that targets PRPF31 or its RNA transcript.
14. The transplant composition of paragraph 12, wherein the vector is selected from the group consisting of: a plasmid and a viral vector.
15. The transplant composition of paragraph 13, wherein the RNAi molecule comprises the nucleic acid sequence of SEQ ID NO: 1.
16. The transplant composition of any one of paragraphs 11-15, wherein the in vitro-differentiated human cardiomyocytes are differentiated from induced pluripotent stem cells (iPSCs) or from embryonic stem cells.
17. The transplant composition of any one of paragraphs 11-16, wherein the cardiomyocytes are differentiated from iPSCs derived from the transplant recipient.
18. A method of transplanting in vitro-differentiated human cardiomyocytes, the method comprising transplanting into cardiac tissue of a subject in vitro-differentiated human cardiomyocytes that have been contacted with an agent that decreases the level or activity of PRPF31.
19. The method of paragraph 18, wherein the contacted cardiomyocytes survive transplanting to a greater extent than cardiomyocytes not contacted with the agent.
20. The method of any one of paragraphs 18-19, wherein the subject has suffered a cardiac infarction.
21. The method of any one of paragraphs 18-20, wherein the agent is a small molecule, a polypeptide, a nucleic acid molecule or a vector comprising a nucleic acid molecule.
22. The method of any one of paragraphs 18-20, wherein the agent comprises or encodes a nucleic acid molecule comprising an antisense sequence, an aptamer or an RNA interference molecule (RNAi) that targets PRPF31 or its RNA transcript.
23. The method of paragraph 21, wherein the vector is selected from the group consisting of: a plasmid and a viral vector.
24. The method of paragraph 22, wherein the RNAi molecule comprises the nucleic acid sequence of SEQ ID NO: 1.
25. The method of any one of paragraphs 18-24, wherein the human cardiomyocytes are differentiated from induced pluripotent stem cells (iPSCs) or from embryonic stem cells.
26. The method of paragraph 25, wherein the iPSCs are derived from the subject.
27. The method of paragraph 25, wherein the iPSCs are derived from a healthy donor.
28. A method of promoting survival and/or engraftment of transplanted human, in vitro-differentiated cardiomyocytes, the method comprising contacting human, in vitro-differentiated cardiomyocytes with an agent that decreases the level or activity of PRPF31, and transplanting the cells into cardiac tissue of a human subject in need thereof
29. The method of any one of paragraphs 28, wherein the subject has suffered a cardiac infarct.
30. The method of any one of paragraphs 28-29, wherein the agent is a small molecule, a polypeptide, a nucleic acid molecule or a vector comprising a nucleic acid molecule.
31. The method of any one of paragraphs 28-30, wherein the agent comprises or encodes a nucleic acid molecule comprising an antisense sequence, an aptamer or an RNA interference molecule (RNAi) that targets PRPF31 or its RNA transcript.
32. The method of paragraph 30, wherein the vector is selected from the group consisting of: a plasmid and a viral vector.
33. The method of paragraph 31, wherein the RNAi molecule comprises the nucleic acid sequence of SEQ ID NO: 1.
34. A method of promoting survival and/or engraftment of transplanted mesoderm lineage cells, the method comprising: administering to a subject in need thereof mesoderm lineage cells contacted or treated with an agent that decreases the level or activity of PRPF31 in the subject.
35. The method of paragraph 34, wherein the mesoderm-derived cells are in vitro differentiated mesoderm lineage cells.
36. The method of paragraph 35, wherein the mesoderm lineage cells are differentiated in vitro from iPS cells or embryonic stem cells.
37. The method of any one of paragraphs 34-36, wherein the agent is a small molecule, a polypeptide, a nucleic acid molecule or a vector comprising a nucleic acid molecule.
38. The method of paragraph 37, wherein the agent comprises or encodes a nucleic acid molecule comprising an antisense sequence, an aptamer or an RNA interference molecule (RNAi) that targets PRPF31 or its RNA transcript.
39. The method of paragraph 37, wherein the vector is selected from the group consisting of: a plasmid and a viral vector.
40. The method of paragraph 38, wherein the RNAi molecule comprises the nucleic acid sequence of SEQ ID NO: 1.
41. The method of any one of paragraphs 36-40, wherein the iPSCs are derived from the subject.
42. The method of any one of paragraphs 36-40, wherein the iPSCs are derived from a healthy donor.
43. The method of any one of paragraphs 34-42, wherein the transplanted mesoderm lineage cells are of a cell type selected from: cardiomyocytes, skeletal muscle cells, smooth muscle cells, kidney cells, endothelial cells, skin cells, adrenal cortex cells, bone cells, white blood cells, and microglial cells.
44. A transplant composition for transplant to a recipient, the composition comprising in vitro-differentiated mesodermal lineage cells that have been contacted or treated with an agent that decreases the level or activity of PRPF31, and a pharmaceutically acceptable carrier.
45. The transplant composition of paragraph 44, wherein the agent is selected from a small molecule, a polypeptide, a nucleic acid molecule or a vector comprising a nucleic acid molecule.
46. The transplant composition of any one of paragraphs 44-45, wherein the agent comprises or encodes a nucleic acid molecule comprising an antisense sequence, an aptamer or an RNA interference molecule (RNAi) that targets PRPF31 or its RNA transcript.
47. The transplant composition of paragraph 45, wherein the vector is selected from the group consisting of: a plasmid and a viral vector.
48. The transplant composition of paragraph 46, wherein the RNAi molecule comprises the nucleic acid sequence of SEQ ID NO: 1.
49. The transplant composition of any one of paragraphs 44-48, wherein the in vitro-differentiated mesodermal lineage cells are differentiated from induced pluripotent stem cells (iPSCs) or from embryonic stem cells.
50. The transplant composition of any one of paragraphs 44-49, wherein the mesodermal lineage cells are differentiated from iPSCs derived from the transplant recipient.
51. A method of transplanting in vitro-differentiated mesodermal lineage cells, the method comprising transplanting into a tissue of a subject in vitro-differentiated mesodermal lineage cells that have been contacted or treated with an agent that decreases the level or activity of PRPF31.
52. The method of paragraph 51, wherein the contacted in vitro-differentiated mesodermal lineage cells survive transplanting to a greater extent than in vitro-differentiated mesodermal lineage cells not contacted with the agent.
53. The method of any one of paragraphs 51-52, wherein the subject has suffered a cardiac infarction.
54. The method of any one of paragraphs 51-53, wherein the agent is a small molecule, a polypeptide, a nucleic acid molecule or a vector comprising a nucleic acid molecule.
55. The method of any one of paragraphs 51-54, wherein the agent comprises or encodes a nucleic acid molecule comprising an antisense sequence, an aptamer or an RNA interference molecule (RNAi) that targets PRPF31 or its RNA transcript.
56. The method of any one of paragraphs 54, wherein the vector is selected from the group consisting of: a plasmid and a viral vector.
57. The method of paragraph 55, wherein the RNAi molecule comprises the nucleic acid sequence of SEQ ID NO: 1.
58. The method of any one of paragraphs 51-57, wherein the mesodermal lineage cells are differentiated from induced pluripotent stem cells (iPSCs) or from embryonic stem cells.
59. The method of paragraph 58, wherein the iPSCs are derived from the subject.
60. The method of paragraph 58, wherein the iPSCs are derived from a healthy donor.
61. The method of any one of paragraphs 51-60, wherein the transplanted mesoderm lineage cells are of a cell type selected from: cardiomyocytes, skeletal muscle cells, smooth muscle cells, kidney cells, endothelial cells, skin cells, adrenal cortex cells, bone cells, white blood cells, and microglial cells.

Unless otherwise explained, all technical and scientific terms used herein have the same meaning as commonly understood by one of ordinary skill in the art to which this disclosure belongs.

It should be understood that this disclosure is not limited to the particular methodology, protocols, and reagents, etc., described herein and as such may vary. The terminology used herein is for the purpose of describing particular embodiments only, and is not intended to limit the scope of the present disclosure, which is defined solely by the claims.

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

### EXAMPLES

### Example 1: Improved Survival of Engrafted Stem-Cell Derived Cardiomyocytes by PRPF31 Gene Expression Knockdown

### BACKGROUND

LaMacchia et al. (2015), describes the effects of knock-down of a set of genes on the survival of C. elegans under hypo-osmotic and hypoxic stress conditions. A candidate list of genes was selected for testing knockdown effects in human pluripotent stem cell-derived cardiomyocytes (hPSC-CM). The six candidate genes were selected based on the following criteria. First, they showed robust effects in the C. elegans model. Second, the human homologs showed high sequence identity to the C. elegans genes. Table 1 below includes the six candidate genes chosen for analysis.

TABLE 1
CANDIDATE GENES

| | | | C. elegans |
|---|---|---|---|
| Gene | Gene Name | Function | Survival |
| CENPC | Centromere Protein C | Cell Division | 40% |
| CSNK2B | Casein Kinase 2, Beta polypeptide | Signal transduction | 57% |
| RUVBL1 | RuvB-like AAA ATPase | Chromatin Remodeling | 47% |
| RCL1 | RNA Terminal Phosphate Cyclase- | Ribosome Biogenesis | 78% |
| | like 1 | | |
| PRPF31 | Pre-mRNA Processing Factor 31 | Spliceosome Activation | 49% |
| GLTSCR2 | Glioma Tumor Suppressor Candidate | Ribosome Biogenesis | 65% |
| | Region 2 | | |
| Control | | | 10% |

Gene Knockdown

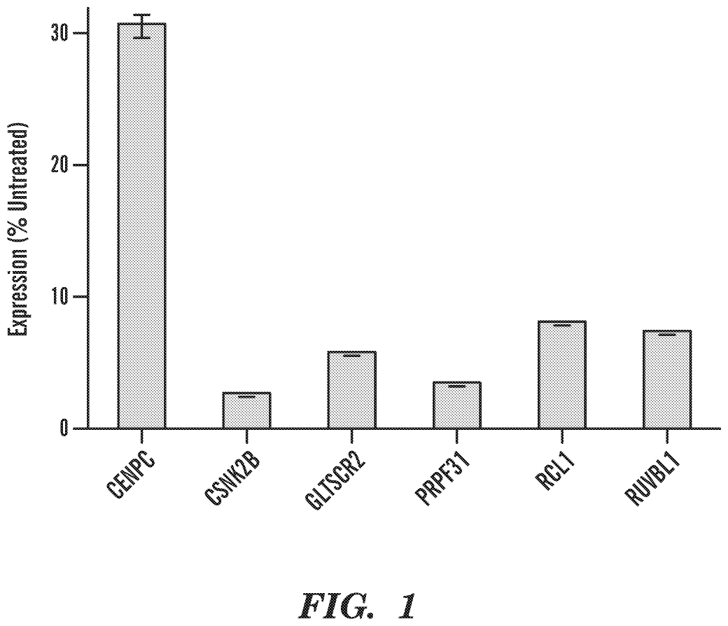

Gene knockdown was executed in hPSC-CM derived from the RUES2 embryonic stem cell line. For each gene of interest, hPSC-CM were transfected with 5 nM siRNA using Lipofectamine RNAiMax (Thermo Fisher) incubation for 48 hours. Controls were untreated or transfected with a negative control scrambled siRNA. The efficiency of knockdown was confirmed by quantitative rtPCR. The resultant cells were cryopreserved for transplantation (FIG. 1).

Transplantation

For transplantation, male NOD scid gamma (NSG) mice were subjected to cardiac infarction by permanent occlusion of the left anterior descending artery. Immediately after occlusion, 2.5×10⁵ cells in 10 μL RPMI culture medium were injected into the left ventricular wall at the site of infarction. Three days post-injection, the mice were sacrificed, and the hearts were collected and snap frozen in liquid nitrogen for subsequent analysis.

Tissue Analysis

DNA from the heart tissue was isolated with a DNeasy Blood and Tissue Kit (Qiagen) according to the manufacturers instructions. The resultant DNA samples were assayed for the presence of human ALU sequence by quantitative PCR using SYBR MasterMix (Applied Biosystems) and the CFX Connect PCR instrument (BioRad). Human ALU element primers were GTC AGG AGA TCG AGA CCA TCC C (forward) and TCC TGC CTC AGC CTC CCA AG (reverse) as described in Robey et al. (2008). 1 to 100,000 pg of human DNA spiked into 100 ng of naïve mouse heart DNA was used to generate a standard curve in each assay.

Results

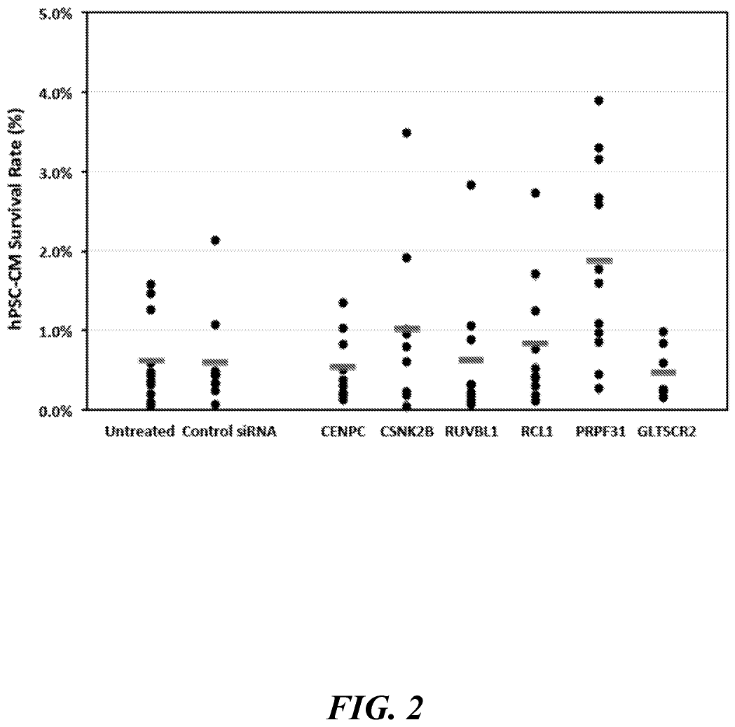

The survival of hPSC-CM with PRPF31 knockdown was increased compared to untreated and control siRNA-treated hPSC-CM (p=0.008 and p=0.007, respectively; unpaired t test) (FIG. 2).

Summary of Results

It is noted that while each of the six different genes showed robust enhancement of survival in C. elegans upon knockdown, only one, PRPF31, provided a benefit to transplanted cardiomyocyte survival in the mouse model. Based on the results, down-regulated PRPF31 expression can improve engraftment/survival of transplanted mammalian cells, such as in vitro-differentiated hPSC-CMs.

### REFERENCES

LaMacchia J C, Frazier H N, III, Roth M B (2015) Glycogen fuels survival during hyposmotic-anoxic stress in Caenorhabditis elegans. Genetics 201:65-74.
Robey T E, Saiget M K, Reinecke H, Murry C E (2008) Systems approaches to preventing transplanted cell death in cardiac repair. J Mol Cell Cardiol 45(4):567-581.

SEQUENCES

```
SEQ ID NO: 1 siRNA Sequence
CGGGAUAAGUACUCAAAGATT
As an alternative, the TT overhang at the 3' end of SEQ ID NO: 1 can be
substituted by a UU (SEQ ID NO: 3).
SEQ ID NO: 2 siRNA Anti-sense strand
UCUUUGAGUACUUAUCCCGGA
SEQ ID NO: 4-Homo sapiens pre-mRNA processing factor 31 (PRPF31), mRNA
NCBI Reference Sequence: NM_015629.4
      1 ggtgagcgac taacgctaga aacagtggtg cgcggagagg agaggcctcg ggatgtctct
    61 ggcagatgag ctcttagctg atctcgaaga ggcagcagaa gaggaggaag gaggaagcta
  121 tggggaggaa gaagaggagc cagcgatcga ggatgtgcag gaggagacac agctggatct
  181 ttccggggat tcagtcaaga ccatcgccaa gctatgggat agtaagatgt ttgctgagat
  241 tatgatgaag attgaggagt atatcagcaa gcaagccaaa gcttcagaag tgatgggacc
  301 agtggaggcc gcgcctgaat accgcgtcat cgtggatgcc aacaacctga ccgtggagat
  361 cgaaaacgag ctgaacatca tccataagtt catccgggat aagtactcaa agagattccc
  421 tgaactggag tccttggtcc ccaatgcact ggattacatc cgcacggtca aggagctggg
  481 caacagcctg gacaagtgca agaacaatga gaacctgcag cagatcctca ccaatgccac
  541 catcatggtc gtcagcgtca ccgcctccac cacccagggg cagcagctgt cggaggagga
  601 gctggagcgg ctggaggagg cctgcgacat ggcgctggag ctgaacgcct ccaagcaccg
  661 catctacgag tatgtggagt cccggatgtc cttcatcgca cccaacctgt ccatcattat
  721 cggggcatcc acggccgcca agatcatggg tgtggccggc ggcctgacca acctctccaa
  781 gatgcccgcc tgcaacatca tgctgctcgg ggcccagcgc aagacgctgt cgggcttctc
  841 gtctacctca gtgctgcccc acaccggcta catctaccac agtgacatcg tgcagtccct
  901 gccaccggat ctgcggcgga aagcggcccg gctggtggcc gccaagtgca cactggcagc
  961 ccgtgtggac agtttccacg agagcacaga agggaaggtg ggctacgaac tgaaggatga
1021 gatcgagcgc aaattcgaca agtggcagga gccgccgcct gtgaagcagg tgaagccgct
1081 gcctgcgccc ctggatggac agcggaagaa gcgaggcggc cgcaggtacc gcaagatgaa
1141 ggagcggctg gggctgacgg agatccggaa gcaggccaac cgtatgagct tcggagagat
1201 cgaggaggac gcctaccagg aggacctggg attcagcctg ggccacctgg gcaagtcggg
1261 cagtgggcgt gtgcggcaga cacaggtaaa cgaggccacc aaggccagga tctccaagac
1321 gctgcagcgg accctgcaga agcagagcgt cgtatatggc gggaagtcca ccatccgcga
1381 ccgctcctcg ggcacggcct ccagcgtggc cttcacccca ctccagggcc tggagattgt
1441 gaacccacag gcggcagaga agaaggtggc tgaggccaac cagaagtatt tctccagcat
1501 ggctgagttc ctcaaggtca agggcgagaa gagtggcctt atgtccacct gaatgactgc
1561 gtgtgtccaa ggtggcttcc cactgaaggg acacagaggt ccagtccttc tgaagggcta
1621 ggatcgggtt ctggcaggga gaacctgccc tgccactggc cccattgctg ggactgccca
1681 gggaggaggc cttggaagag tccggcctgg cctcccccag gaccgagatc accgcccagt
1741 atgggctaga gcaggtcttc atcatgcctt gtctttttta actgagaaag gagatttttt
1801 gaaaagagta caattaaaag gacattgtca aga
SEQ ID NO: 5-U4/U6 small nuclear ribonucleoprotein Prp31 [Homo sapiens]
NCBI Reference Sequence: XP_006723200.1
      1 msladellad leeaaeeeeg gsygeeeeep aiedvqeetq ldlsgdsvkt iaklwdskmf
    61 aeimmkieey iskqakasev mgpveaapey rvivdannit veienelnii hkfirdkysk
  121 rfpeleslvp naldyirtvk elgnsldkck nnenlqqilt natimvvsvt asttqgqqls
  181 eeelerleea cdmalelnas khriyeyves rmsfiapnls iiigastaak imgvaggltn
  241 lskmpacnim llgagrktls gfsstsvlph tgyiyhsdiv qslppdlrrk aarlvaakct
  301 laarvdsfhe stegkvgyel kdeierkfdk wqepppvkqv kplpapldgq rkkrggrryr
  361 kmkerlglte irkganrmsf geieedayqe dlgfslghlg ksgsgrvrqt qvneatkari
  421 sktlqrtlqk qsvvyggkst irdrssgtas svaftplqgl eivnpqaaek kvaeanqkyf
  481 ssmaeflkvk geksglmst
```

1. A composition comprising human cells differentiated in vitro from stem cells and an agent that decreases the level or activity of Pre-mRNA Processing Factor 31 (PRPF31).

2. (canceled)

3. The composition of claim 1, wherein the cells differentiated in vitro from stem cells are of a mesodermal lineage.

4. The composition of claim 3, wherein the in vitro-differentiated cells are of a cell type selected from: cardiomyocytes, skeletal muscle cells, smooth muscle cells, kidney cells, endothelial cells, skin cells, adrenal cortex cells, bone cells, white blood cells, and microglial cells.

5. The composition of claim 1, wherein the in vitro-differentiated human cells are differentiated from induced pluripotent stem cells (iPSCs) or from embryonic stem cells.

6. The composition of claim 1, wherein the stem cells are derived from a healthy subject.

7. The composition of claim 1, wherein the agent is a small molecule, a polypeptide, a nucleic acid molecule or a vector comprising a nucleic acid molecule.

8. The composition of claim 7, wherein the agent comprises or encodes a nucleic acid molecule comprising an antisense sequence, an aptamer or an RNA interference molecule (RNAi) that targets PRPF31 or its RNA transcript.

9. (canceled)

10. The composition of claim 8, wherein the RNAi molecule comprises the nucleic acid sequence of SEQ ID NO: 1.

11. A transplant composition for transplant to a recipient, the composition comprising in vitro-differentiated human cardiomyocytes that have been contacted with an agent that decreases the level or activity of PRPF31, and a pharmaceutically acceptable carrier.

12. The transplant composition of claim 11, wherein the agent is selected from a small molecule, a polypeptide, a nucleic acid molecule or a vector comprising a nucleic acid molecule.

13. The transplant composition of claim 11, wherein the agent comprises or encodes a nucleic acid molecule comprising an antisense sequence, an aptamer or an RNA interference molecule (RNAi) that targets PRPF31 or its RNA transcript.

14. (canceled)

15. The transplant composition of claim 13, wherein the RNAi molecule comprises the nucleic acid sequence of SEQ ID NO: 1.

16. The transplant composition of claim 11, wherein the in vitro-differentiated human cardiomyocytes are differentiated from induced pluripotent stem cells (iPSCs) or from embryonic stem cells.

17. The transplant composition of claim 11, wherein the cardiomyocytes are differentiated from iPSCs derived from the transplant recipient.

18. A method of transplanting in vitro-differentiated human cardiomyocytes, the method comprising transplanting into cardiac tissue of a subject in vitro-differentiated human cardiomyocytes that have been contacted with an agent that decreases the level or activity of PRPF31.

19. The method of claim 18, wherein the contacted cardiomyocytes survive transplanting to a greater extent than cardiomyocytes not contacted with the agent.

20. The method of claim 18, wherein the subject has suffered a cardiac infarction.

21. The method of claim 18, wherein the agent is a small molecule, a polypeptide, a nucleic acid molecule or a vector comprising a nucleic acid molecule.

22. The method of claim 18, wherein the agent comprises or encodes a nucleic acid molecule comprising an antisense sequence, an aptamer or an RNA interference molecule (RNAi) that targets PRPF31 or its RNA transcript.

23. (canceled)

24. The method of claim 22, wherein the RNAi molecule comprises the nucleic acid sequence of SEQ ID NO: 1.

25.-61. (canceled)
